SEQUENCE LISTING (25 sequences)

SEQ ID NO: 1 - protein, 15 aa, Artificial Sequence
  CDRL1 -  hPD-1.09A
RASKGVSTSGYSYLH

SEQ ID NO: 2 - protein, 7 aa, Artificial Sequence
  CDRL2 hPD-1.09A
LASYLES

SEQ ID NO: 3 - protein, 9 aa, Artificial Sequence
  CDRL3 hPD-1.09A
QHSRDLPLT

SEQ ID NO: 4 - protein, 111 aa, Artificial Sequence [sequence omitted]

SEQ ID NO: 5 - protein, 218 aa, Artificial Sequence
  mature K09A light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  CDRH1 hPD-1.09A
NYYMY

SEQ ID NO: 7 - protein, 17 aa, Artificial Sequence
  CDRH2 - hPD-1.09A
GINPSNGGTNFNEKFKN

SEQ ID NO: 8 - protein, 11 aa, Artificial Sequence
  CDRH3 hPD-1.09A
RDYRFDMGFDY

SEQ ID NO: 9 - protein, 120 aa, Artificial Sequence [sequence omitted]

SEQ ID NO: 10 - protein, 447 aa, Artificial Sequence
  mature h109A heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 15 aa, Artificial Sequence
  CDRL1 hPD-1.08A
RASKSVSTSGFSYLH

SEQ ID NO: 12 - protein, 7 aa, Artificial Sequence
  CDRL2 hPD1.08A
LASNLES

SEQ ID NO: 13 - protein, 9 aa, Artificial Sequence
  CDRL3 hPD-1.08A
QHSWELPLT

SEQ ID NO: 14 - protein, 5 aa, Artificial Sequence
  CDRH1 hPD-1.08A
SYYLY

SEQ ID NO: 15 - protein, 17 aa, Artificial Sequence
  CDRH2 hPD-1.08A
GVNPSNGGTNFSEKFKS

SEQ ID NO: 16 - protein, 11 aa, Artificial Sequence
  CDRH3 hPD-1.08A
RDSNYDGGFDY

SEQ ID NO: 17 - protein, 290 aa, Artificial Sequence
  mature human PD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 18 - protein, 112 aa, Artificial Sequence
  20C3 light chain mature variable region
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCQQSYDVVTFGAGTKLELK

SEQ ID NO: 19 - protein, 122 aa, Artificial Sequence
  20C3 heavy chain mature variable region
QVQVQQSGAELAEPGASVKMSCKASGYIFTSYWMHWLKQRPGQGLEWIGYINPSSDYNEYSEKFMDKATLTADKASTTAYMQLISLTSEDSAVYYCARSGWLVHGDYYFDYWGQGTTLTVSS

SEQ ID NO: 20 - protein, 112 aa, Artificial Sequence
  22C3 light chain mature variable region
DIVMSQSPSSLAVSAGEKVTMTCKSSQSLLHTSTRKNYLAWYQQKPGQSPKLLIYWASTRESGVPDRFTGSGSGTDFTLTISSVQAEDLAVYYCKQSYDVVTFGAGTKLELK

SEQ ID NO: 21 - protein, 122 aa, Artificial Sequence
  22C3 heavy chain mature variable region
QVHLQQSGAELAKPGASVKMSCKASGYTFTSYWIHWIKQRPGQGLEWIGYINPSSGYHEYNQKFIDKATLTADRSSSTAYMHLTSLTSEDSAVYYCARSGWLIHGDYYFDFWGQGTTLTVSS

SEQ ID NO: 22 - protein, 111 aa, Artificial Sequence
  mature K09A light chain variable region
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 23 - protein, 111 aa, Artificial Sequence
  mature k09A light chain variable region
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 24 - protein, 218 aa, Artificial Sequence
  mature k09A light chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 218 aa, Artificial Sequence
  mature K09A light chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0074983 A1
(43) Pub. Date: Mar. 6, 2025

(54) METHODS FOR TREATING CANCER WITH SUBCUTANEOUS ADMINISTRATION OF ANTI-PD1 ANTIBODIES

(71) Applicants: Mallika LALA, Rahway, NJ (US); Carolina DE MIRANDA SILVA, Rahway, NJ (US); Ferdous GHEYAS, Rahway, NJ (US); Yogita KRISHNA MACHARI, Kenilworth, NJ (US); Elliot Keith CHARTASH, Rahway, NJ (US); Lokesh JAIN, Edison, NJ (US); Venkata Naga Ratna Pavan Kumar VADDADY, Doylestown, PA (US); MERCK SHARP & DOHME LLC, Rahway, NJ (US)

(72) Inventors: Mallika LALA, West New York, NJ (US); Carolina DE MIRANDA SILVA, Rahway, NJ (US); Ferdous GHEYAS, Edison, NJ (US); Yogita KRISHNAMACHARI, Scotch Plains, NJ (US); Elliot Keith CHARTASH, Basking Ridge, NJ (US); Lokesh JAIN, Edison, NJ (US); Venkata Naga Ratna Pavan Kumar VADDADY, Doylestown, PA (US)

(73) Assignee: MERCK SHARP & DOHME LLC, Rahway, NJ (US)

(21) Appl. No.: 18/554,213

(22) PCT Filed: Apr. 4, 2022
(86) PCT No.: PCT/US2022/023250

§ 371 Date: Oct. 6, 2023

Related U.S. Application Data
(60) Provisional application No. 63/172,299, filed on Apr. 8, 2021.

(51) Int. Cl. C07K 16/28

(52) U.S. Cl. CPC C07K 16/2818; C07K 2317/565

(57) ABSTRACT

The invention relates to methods for treating cancer in a patient comprising subcutaneously administering a PD-1 antagonist, e.g., an anti-PD-1 antibody, or antigen binding fragment thereof, (e.g. pembrolizumab), in specific amounts to the patient. In some embodiments, the administration occurs about every three weeks. In some embodiments, the amount of anti-PD-1 antibody, or antigen binding fragment thereof, is about 280 mg to about 450 mg. In certain embodiments, the PD-1 antagonist is pembrolizumab, or an antigen binding fragment thereof. Also provided are compositions and kits formulated for subcutaneous administration comprising a dosage of an anti-PD-1 antibody, or antigen-binding fragment thereof, and uses thereof for treating cancer.

### FIELD OF THE INVENTION

The invention relates to therapies useful for the treatment of cancer. In particular, the invention relates to a method for treating cancer which comprises administering to a patient in need thereof an anti-PD-1 antibody, or antigen binding fragment thereof, using the dosage regimens specified herein.

### CROSS-REFERENCE TO RELATED APPLICATIONS

This application claims the benefit of U.S. Ser. No. 63/172,299, filed Apr. 8, 2021, the contents of which are herein incorporated by reference in its entirety.

### REFERENCE TO SEQUENCE LISTING SUBMITTED ELECTRONICALLY

The sequence listing of the present application is submitted electronically via EFS-Web as an ASCII formatted sequence listing with a file name “25131WOPCT-SEQLIST-21mar.2022.TXT”, creation date of Apr. 6, 2021, and a size of 23.5 kb. This sequence listing submitted via EFS-Web is part of the specification and is herein incorporated by reference in its entirety.

### BACKGROUND OF THE INVENTION

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (Sharpe et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong et al., Nat Med. 8(8):793-800 (2002); Yang et al. Invest Ophthalmol Vis Sci. 49: 2518-2525 (2008); Ghebeh et al. Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953 (2005); Inman et al., Cancer 109: 1499-1505 (2007); Shimauchi et al. Int. J. Cancer 121:2585-2590 (2007); Gao et al. Clin. Cancer Research 15: 971-979 (2009); Nakanishi J. Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T cells in breast cancer and melanoma (Ghebeh et al., BMC Cancer. 2008 8:5714-15 (2008); Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T cells to attenuate T cell activation and evasion of immune surveillance, thereby contributing to an impaired immune response against the tumor.

Immune checkpoint therapies targeting the PD-1 axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; Wolchok et al., N Engl J Med 2013, 369: 122-33). Immune therapies targeting the PD-1 axis include monoclonal antibodies directed to the PD-1 receptor (KEYTRUDA™ (pembrolizumab), Merck and Co., Inc., Kenilworth, NJ, USA and OPDIVO™ (nivolumab), Bristol-Myers Squibb Company, Princeton, NJ, USA) and also those that bind to the PD-L1 ligand (MPDL3280A; TECENTRIQ™ (atezolizumab), Genentech, San Francisco, CA, USA; IMFINZI™ (durvalumab), AstraZeneca Pharmaceuticals LP, Wilmington, DE; BAVENCIO™ (avelumab), Merck KGaA, Darmstadt, Germany). Both therapeutic approaches have demonstrated anti-tumor effects in numerous cancer types.

Currently approved anti-PD-1 antibody treatments for use in multiple cancer indications are administered as an IV infusion at a dose of (i) either 200 mg or 2 mg/kg Q3W or (ii) 400 mg Q6W. It would be beneficial to develop a dosing schedule that allows for the administration of a safe and effective subcutaneous dose of an anti-PD-1 antibody that provides comparable exposure of the approved IV infusion dose. An alternative to IV infusions, such as a subcutaneous administration, would provide convenience and flexibility to patients, reduce patient time in the treatment room, and shorten the time needed by providers to administer the treatment.

### SUMMARY OF THE INVENTION

The invention provides alternative, convenient, cost efficient, subcutaneous dosing regimens for treating a cancer patient with an anti-PD-1 antibody, or antigen-binding fragment thereof, wherein the dosing schedule is expected to provide a safe and effective dose of the anti-PD-1 antibody, or antigen-binding fragment thereof. Specifically, the invention provides a method of treating cancer in a human patient comprising subcutaneously administering about 280 mg to about 450 mg of an anti-PD-1 antibody, or antigen binding fragment thereof, to the patient every three weeks; wherein the anti-PD-1 antibody or antigen-binding fragment thereof comprises (a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively; or (b) light chain CDRs LC-CDR1, LC-CDR2 and LC-CDR3 comprising a sequence of amino acids as set forth in SEQ ID NOs: 11, 12 and 13, respectively, and heavy chain CDRs HC-CDR1, HC-CDR2 and HC-CDR3 comprising a sequence of amino acids as set forth in SEQ ID NOs: 14, 15 and 16, respectively. In some embodiments, the antibody or antigen binding fragment thereof is administered every three weeks. In embodiments of the invention, the antibody or antigen-binding fragment is pembrolizumab or an antigen-binding fragment thereof. In a further embodiment, the anti-PD-1 antibody is pembrolizumab.

The invention also provides a method of treating cancer in a human patient comprising subcutaneously administering to the patient approximately every three weeks a dose of an anti-PD-1 antibody, or antigen binding fragment thereof, that is at least 1.6 times higher than a 200 mg or a 2 mg/kg dose of the anti-PD-1 antibody, or antigen binding fragment thereof, administered by an IV route of administration approximately every three weeks, wherein the anti-PD-1 antibody or antigen-binding fragment thereof comprises (a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively; or (b) light chain CDRs LC-CDR1, LC-CDR2 and LC-CDR3 comprising a sequence of amino acids as set forth in SEQ ID NOs: 11, 12 and 13, respectively, and heavy chain CDRs HC-CDR1, HC-CDR2 and HC-CDR3 comprising a sequence of amino acids as set forth in SEQ ID NOs: 14, 15 and 16, respectively. In some embodiments, the antibody or antigen binding fragment thereof is administered every three weeks. In embodiments of the invention, the antibody or antigen-binding fragment is pembrolizumab or an antigen-binding fragment thereof. In a further embodiment, the anti-PD-1 antibody is pembrolizumab.

The invention also provides a method of treating cancer in a human patient comprising subcutaneously administering to the patient approximately every three weeks a dose of an anti-PD-1 antibody, or antigen binding fragment thereof, that is at least 1.6 times higher than a 200 mg or a 2 mg/kg dose of the anti-PD-1 antibody, or antigen binding fragment thereof, wherein the anti-PD-1 antibody or antigen-binding fragment thereof comprises (a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively; or (b) light chain CDRs LC-CDR1, LC-CDR2 and LC-CDR3 comprising a sequence of amino acids as set forth in SEQ ID NOs: 11, 12 and 13, respectively, and heavy chain CDRs HC-CDR1, HC-CDR2 and HC-CDR3 comprising a sequence of amino acids as set forth in SEQ ID NOs: 14, 15 and 16, respectively. In some embodiments, the antibody or antigen binding fragment thereof is administered every three weeks. In some embodiments of the invention, the antibody or antigen-binding fragment is pembrolizumab or an antigen-binding fragment thereof. In a further embodiment, the anti-PD-1 antibody is pembrolizumab. In embodiments of the methods described above, the bioavailability of the anti-PD-1 antibody, or antigen binding fragment thereof, is at least 63%. In embodiments of the methods described above, the bioavailability of the anti-PD-1 antibody, or antigen binding fragment thereof, is at least 64%. In embodiments of the methods described above, the bioavailability of the anti-PD-1 antibody, or antigen binding fragment thereof, is at least 66%.

In embodiments of the invention, the amount of anti-PD-1 antibody or antigen-binding fragment thereof administered to the patient is from 320 mg to 420 mg, from 340 mg to 420 mg, from 345 mg to 415 mg, from 350 mg to 410 mg, from 355 mg to 405 mg, from 360 mg to 400 mg, from 365 mg to 395 mg, from 370 mg to 390 mg, from 375 mg to 385 mg, or from 379 mg to 381 mg.

In embodiments of the invention, the amount of anti-PD-1 antibody or antigen-binding fragment thereof administered to the patient is from about 280 mg to about 450 mg. In further embodiments, the amount of antibody or antigen-binding fragment is about 300 mg to about 450 mg. In further embodiments, the amount of antibody or antigen-binding fragment is about 320 mg to about 450 mg. In further embodiments, the amount of antibody or antigen-binding fragment is about 340 mg to about 450 mg. In further embodiments, the amount of antibody or antigen-binding fragment is about 360 mg to about 450 mg. In further embodiments, the amount of antibody or antigen-binding fragment is about 370 mg to about 450 mg. In further embodiments, the amount of antibody or antigen-binding fragment is about 375 mg to about 450 mg. In further embodiments, the amount of antibody or antigen-binding fragment is about 300 mg to about 430 mg. In further embodiments, the amount of antibody or antigen-binding fragment is about 320 mg to about 430 mg. In further embodiments, the amount of antibody or antigen-binding fragment is about 340 mg to about 430 mg. In further embodiments, the amount of antibody or antigen-binding fragment is about 360 mg to about 430 mg. In further embodiments, the amount of antibody or antigen-binding fragment is about 370 mg to about 430 mg. In further embodiments, the amount of antibody or antigen-binding fragment is about 375 mg to about 430 mg. In further embodiments, the amount of antibody or antigen-binding fragment is about 320 mg to about 420 mg. In further embodiments, the amount of antibody or antigen-binding fragment is about 340 mg to about 420 mg. In further embodiments, the amount of antibody or antigen-binding fragment is about 360 mg to about 420 mg. In further embodiments, the amount of antibody or antigen-binding fragment is about 370 mg to about 420 mg. In further embodiments, the amount of antibody or antigen-binding fragment is about 345 mg to about 415 mg. In further embodiments, the amount of antibody or antigen-binding fragment is about 300 mg to about 410 mg. In further embodiments, the amount of antibody or antigen-binding fragment is about 320 mg to about 410 mg. In further embodiments, the amount of antibody or antigen-binding fragment is about 340 mg to about 410 mg. In further embodiments, the amount of antibody or antigen-binding fragment is about 350 mg to about 410 mg. In further embodiments, the amount of antibody or antigen-binding fragment is about 360 mg to about 410 mg. In further embodiments, the amount of antibody or antigen-binding fragment is about 370 mg to about 410 mg. In further embodiments, the amount of antibody or antigen-binding fragment is about 375 mg to about 410 mg. In further embodiments, the amount of antibody or antigen-binding fragment is about 355 mg to about 405 mg. In further embodiments, the amount of antibody or antigen-binding fragment is about 360 mg to about 400 mg. In further embodiments, the amount of antibody or antigen-binding fragment is about 365 mg to about 395 mg. In further embodiments, the amount of antibody or antigen-binding fragment is about 300 mg to about 390 mg. In further embodiments, the amount of antibody or antigen-binding fragment is about 320 mg to about 390 mg. In further embodiments, the amount of antibody or antigen-binding fragment is about 340 mg to about 390 mg. In further embodiments, the amount of antibody or antigen-binding fragment is about 360 mg to about 390 mg. In further embodiments, the amount of antibody or antigen-binding fragment is about 370 mg to about 390 mg. In further embodiments, the amount of antibody or antigen-binding fragment is about 375 mg to about 390 mg. In further embodiments, the amount of antibody or antigen-binding fragment is about 365 mg to about 395 mg. In further embodiments, the amount of antibody or antigen-binding fragment is about 375 mg to about 385 mg. In further embodiments, the amount of antibody or antigen-binding fragment is about 379 mg to about 381 mg. In further embodiments, the amount of antibody or antigen-binding fragment is about 380 mg. In further embodiments, the amount of antibody or antigen-binding fragment is 380 mg.

In one embodiment, the amount of antibody or antigen binding fragment thereof administered is 280 mg. In one embodiment, the amount of antibody or antigen binding fragment thereof administered is 285 mg. In another embodiment, the amount of antibody or antigen binding fragment thereof administered is 320 mg. In another embodiment, the amount of antibody or antigen binding fragment thereof administered is 340 mg. In another embodiment, the amount of antibody or antigen binding fragment thereof administered is 360 mg. In another embodiment, the amount of antibody or antigen binding fragment thereof administered is 370 mg. In another embodiment, the amount of antibody or antigen binding fragment thereof administered is 380 mg. In another embodiment, the amount of antibody or antigen binding fragment thereof administered is 400 mg. In another embodiment, the amount of antibody or antigen binding fragment thereof administered is 420 mg.

In all of the above treatment methods, compositions and uses herein, the anti-PD-1 antibody or antigen-binding fragment inhibits the binding of PD-L1 to PD-1, and preferably also inhibits the binding of PD-L2 to PD-1. In some embodiments of the treatment methods, compositions and uses of the invention, the anti-PD-1 antibody or antigen-binding fragment is a monoclonal antibody, which specifically binds to PD-1 and blocks the binding of PD-L1 to PD-1. In one particular embodiment, the anti-PD-1 antibody comprises a heavy chain and a light chain, wherein the light and heavy chains comprise the amino acid sequences shown in FIG. 1 (SEQ ID NO:5 and SEQ ID NO:10).

In some embodiments of any of the above treatment methods, compositions and uses, the cancer expresses one or both of PD-L1 and PD-L2. In some embodiments, PD-L1 expression is present or elevated in the cancer.

### BRIEF DESCRIPTION OF THE DRAWINGS

FIG. 1 shows amino acid sequences of the light chain and heavy chain for an exemplary anti-PD-1 monoclonal antibody useful in the invention (SEQ ID NOs: 5 and 10, respectively). Light chain and heavy chain variable regions are underlined (SEQ ID NOs: 4 and 9, respectively) and CDRs are bold.

FIG. 2 shows the observed mean PK profiles for pembrolizumab SC 285 mg and pembrolizumab IV 200 mg in cycle 1. Error bars represent the standard error of the mean. The SC values represent the observed mean PK profile after the first pembrolizumab 285 mg SC dose (both formulation and strengths). The IV values represent the observed mean PK profile after the first pembrolizumab 200 mg IV dose.

FIG. 3A shows a population mean C_{trough} across various SC doses using PK model-based simulations at cycle 1.

FIG. 3B shows a population mean C_{trough} across various SC doses using PK model-based simulations at steady state.

FIG. 4A shows the distribution (median, 5^{th}, 25^{th}, 75^{th}, and 95^{th} percentiles) of C_{trough} at cycle 1 using PK model-based simulations at a dose of 380 mg SC and 200 mg IV of pembrolizumab.

FIG. 4B shows the distribution (median, 5^{th}, 25^{th}, 75^{th}, and 95^{th} percentiles) of C_{trough} at steady state (cycle 6) using PK model-based simulations at a dose of 380 mg SC and 200 mg IV of pembrolizumab.

FIG. 5 shows a 90% CI of SC/IV C_{trough} from cycles 1 to 6 (steady-state) using PK model-based simulations for a NSCLC population at a dose of 380 mg SC and 200 mg IV of pembrolizumab.

FIG. 6 shows a 90% CI of SC/IV AUC_{0-3 wks} from cycles 1 to 6 (steady-state) using PK model-based simulations for a NSCLC population at a dose of 380 mg SC and 200 mg IV of pembrolizumab.

FIG. 7 shows the study design of a Phase III study described in Example 3.

### DETAILED DESCRIPTION OF THE INVENTION

The invention provides methods of treatment (e.g., methods of treating cancer) comprising subcutaneous administration of specified dosages of an anti-PD-1 antibody (e.g., pembrolizumab) or antigen-binding fragment thereof. Such administration is expected to provide a safe and effective dose of the anti-PD-1 antibody or antigen-binding fragment thereof. Also provided are compositions and kits formulated for subcutaneous administration comprising a dosage of an anti-PD-1 antibody, or antigen-binding fragment thereof, and uses thereof for treating cancer. In certain embodiments of the invention, the anti-PD-1 antibody is pembrolizumab or an antigen binding fragment of pembrolizumab.

### I. ABBREVIATIONS AND DEFINITIONS

As used throughout the specification and appended claims, the following abbreviations apply:
AUC area under the concentration-time curve
AUCss area under the concentration-time curve at steady state
CDR complementarity determining region
Cl confidence interval
CL clearance
Cmax,ss peak concentrations at steady state
CPS combined positive score
CV coefficient of variation of between-subject distributions of parameters;
ECOG Eastern Cooperative Oncology Group
eGFR: estimated glomerular filtration rate
α-R exposure (concentration)-response
F: bioavailability;
FFPE formalin-fixed paraffin-embedded
FR framework region
GM geometric mean
HCC hepatocellular carcinoma
HNSCC head and neck squamous cell cancer
HL Hodgkin lymphoma
IgG immunoglobulin G
IHC immunohistochemistry or immunohistochemical
IMAX: maximum effect of time on CL
IV intravenous
ka: first order absorption rate constant
LPS lymphoma proportion score
mAb monoclonal antibody
MCC Merkel cell carcinoma
MEL melanoma
MMR mismatch repair
MPS modified proportion score
MRI magnetic resonance imaging
MSI-H microsatellite instability-high
NCI National Cancer Institute
NSCLC non-small cell lung cancer
OS overall survival
PD-1 programmed death 1 (a.k.a. programmed cell death-1 and
programmed death receptor 1)
PD-L1 programmed cell death 1 ligand 1
PD-L2 programmed cell death 1 ligand 2
PFS progression free survival
PK pharmacokinetic
Q intercompartmental clearance
Q2W one dose every two weeks
Q3W one dose every three weeks
Q6W one dose every six weeks
RCC renal cell carcinoma
RSE relative standard error
SC subcutaneous
TI₅₀ time at which 50% of maximum effect on clearance has been achieved;
t_{lag} lag time for absorption
TPS tumor proportion score
Vc central volume of distribution
V_{H} immunoglobulin heavy chain variable region
V_{L} immunoglobulin light chain variable region
Vp peripheral volume of distribution

Presented population parameter estimates exclude effects of covariates; therefore, such estimates apply to a hypothetical typical patient with average characteristics.

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,”, “an,” and “the” include the plural reference unless the context clearly dictates otherwise. Reference to “or” indicates either or both possibilities unless the context clearly dictates one of the indicated possibilities. In some cases, “and/or” was employed to highlight either or both possibilities.

The term “about”, when modifying the quantity (e.g., mg) of a substance or composition, or the value of a parameter characterizing a step in a method, or the like, refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of ±0.1%, +0.5%, +1%, +2%, ±3%, ±4%, ±5%, ±6%, ±7%, ±8%, ±9%, +10% or +11%. In specific embodiments, when referring to the dosage of “about 380 mg,” the dosage can be, for example, from 340 mg to 420 mg, from 345 mg to 415 mg, from 350 mg to 410 mg, from 355 mg to 405 mg, from 360 mg to 400 mg, from 365 mg to 395 mg, from 370 mg to 390 mg, from 375 mg to 385 mg, or from 379 to 381 mg. In alternative embodiments, the dosage can be 360 mg, 365 mg, 370 mg, 375 mg, 379 mg, 379.5 mg, 380 mg, 385 mg, 390 mg, 395 mg, 400 mg, 405 mg, 410 mg, 415 mg, or 420 mg. When referring to the amount of time between administrations in a therapeutic treatment regimen (i.e., amount of time between administrations of the anti-PD-1 antibody or antigen binding fragment thereof, e.g. “about 3 weeks,” which is used interchangeably herein with “approximately every three weeks”), “about” refers to the stated time±a variation that can occur due to patient/clinician scheduling and availability around the 3-week target date. For example, “about 3 weeks” can refer to 3 weeks±5 days, 3 weeks±4 days, 3 weeks±3 days, 3 weeks±2 days or 3 weeks±1 day, or may refer to 2 weeks, 2 days through 3 weeks, 5 days.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Treat” or “treating” a cancer, as used herein, means to administer an anti-PD-1 antibody, or antigen-binding fragment, to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, decreasing the number of one or more tumor markers, halting or delaying the growth of a tumor or blood cancer or progression of disease associated with PD-1 binding to its ligands PD-L1 and/or PD-L2 (“PD-1-related disease”) such as cancer, stabilization of PD-1-related disease, inhibiting the growth or survival of tumor cells, eliminating or reducing the size of one or more cancerous lesions or tumors, decreasing the level of one or more tumor markers, ameliorating or abrogating the clinical manifestations of PD-1-related disease, reducing the severity or duration of the clinical symptoms of PD-1-related disease such as cancer, prolonging the survival of a patient relative to the expected survival in a similar untreated patient, and inducing complete or partial remission of a cancerous condition or other PD-1 related disease.

Positive therapeutic effects in cancer can be measured in a number of ways (See, W. A. Weber, J. Nucl. Med. 50:1S-iOS (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a therapeutically effective amount is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the treatment methods, compositions and uses of the invention may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

Unless otherwise indicated, an “antibody fragment” or “antigen binding fragment” refers to antigen binding fragments of antibodies, i.e. antibody fragments that retain the ability to specifically bind to the antigen bound by the full-length antibody, e.g. fragments that retain one or more CDR regions, e.g. three heavy chain CDRs and three light chain CDRs. Examples of antibody binding fragments include, but are not limited to, Fab, Fab′, F(ab′)₂, and Fv fragments.

“Anti-PD-1 antibody” as used in any of the treatment methods, refers to compositions and uses of the invention include monoclonal antibodies (mAb), or antigen binding fragments thereof, which specifically bind to human PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, compositions and uses of the invention in which a human individual is being treated, the anti-PD-1 antibody, or antigen binding fragment thereof, is a PD-1 antagonist that blocks binding of human PD-L1 to human PD-1, or blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. An anti-PD-1 antibody may be a human antibody, a humanized antibody or a chimeric antibody, and may include a human constant region. In some embodiments the human constant region is selected from the group consisting of IgG1, IgG2, IgG3 and IgG4 constant regions, and in particular embodiments, the human constant region is an IgG1 or IgG4 constant region. In some embodiments, the antigen binding fragment is selected from the group consisting of Fab, Fab′-SH, F(ab′)₂, scFv and Fv fragments.

“AUCss,” “and “Cmax,ss” are pharmacokinetic measures of the systemic exposure to the drug (e.g. pembrolizumab) in humans after its administration, and are typically considered drivers of drug efficacy. “AUCss” represent the average exposure over a dosing interval. “Cmax,ss” is the maximum or highest (peak) drug concentration observed soon after its administration. In the specific case of pembrolizumab, which is administered as a subcutaneous injection, the peak concentration occurs immediately after end of infusion. Cmax,ss is a metric that is typically considered a driver of driver safety.

“Biotherapeutic agent” means a biological molecule, such as an antibody or fusion protein, that blocks ligand/receptor signaling in any biological pathway that supports tumor maintenance and/or growth or suppresses the anti-tumor immune response.

The term “buffer” encompasses those agents which maintain the solution pH of the formulations of the invention in an acceptable range, or, for Lyophilized formulations of the invention, provide an acceptable solution pH prior to lyophilization. The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

“C_{trough}” is the trough concentration achieved at the end of the dosing interval. The SC:IV C_{trough} ratio is the ratio of the C_{trough} achieved with the SC dose (e.g., a 380 mg SC dose of pembrolizumab) relative to an IV dose (e.g., a 200 mg IV dose of pembrolizumab) at the end of the same dosing interval.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include, but are not limited to, squamous cell carcinoma, myeloma, small-cell lung cancer, non-small cell lung cancer, glioma, Hodgkin's lymphoma, non-Hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Additional cancers that may be treated in accordance with the invention include those characterized by elevated expression of one or both of PD-L1 and PD-L2 in tested tissue samples.

“CDR” or “CDRs” means complementarity determining region(s) in an immunoglobulin variable region, generally defined using the Kabat numbering system.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth. Chemotherapeutic agents useful in the treatment methods of the invention include cytostatic and/or cytotoxic agents.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in the art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Diagnostic anti-PD-L monoclonal antibody” means a mAb which specifically binds to the mature form of the designated PD-L (PD-L1 or PD-L2) that is expressed on the surface of certain mammalian cells. A mature PD-L lacks the presecretory leader sequence, also referred to as leader peptide. The terms “PD-L” and “mature PD-L” are used interchangeably herein, and shall be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence: MRIFAVFIFMTYWHLLNAFTVTVPKDLYVVEYGSNMTIECKFPVEKQLDLAALIVYWE MEDKNIIQFVHGEEDLKVQHSSYRQRARLLKDQLSLGNAALQITDVKLQDAGVYRCMI SYGGADYKRITVKVNAPYNKINQRILVVDPVTSEHELTCQAEGYPKAEVIWTSSDHQVL SGKTTTTNSKREEKLFNVTSTLRINTTTNEIFYCTFRRLDPEENHTAELVIPELPLAHPPNE RTHLVILGAILLCLGVALTFIFRLRKGRMMDVKKCGIQDTNSKKQSDTHLEET (SEQ ID NO:17).

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in WO 2014/100079. These antibodies comprise the light chain and heavy chain variable region amino acid sequences shown in Table 2 below:

TABLE 2
Monoclonal Antibodies 20C3 and 22C3
20C3 Light Chain Mature Variable Region

| | |
|---|---|
| DIVMSQSPSSLAVSAGEKVTMSCKSSQSLLNSRTRKNYLAWY | SEQ ID |
| QQKPGQSPKLLIYWASTRESGVPDRFTGSGSGTDFTLTISSVQ | NO: 18 |
| AEDLAVYYCQQSYDVVTFGAGTKLELK | |

20C3 Heavy Chain Mature Variable Region

| | |
|---|---|
| QVQVQQSGAELAEPGASVKMSCKASGYIFTSYWMHWLKQR | SEQ ID |
| PGQGLEWIGYINPSSDYNEYSEKFMDKATLTADKASTTAYM | NO: 19 |
| QLISLTSEDSAVYYCARSGWLVHGDYYFDYWGQGTTLTVSS | |

22C3 Light Chain Mature Variable Region

| | |
|---|---|
| DIVMSQSPSSLAVSAGEKVTMTCKSSQSLLHTSTRKNYLAWY | SEQ ID |
| QQKPGQSPKLLIYWASTRESGVPDRFTGSGSGTDFTLTISSVQ | NO: 20 |
| AEDLAVYYCKQSYDVVTFGAGTKLELK | |

22C3 Heavy Chain Mature Variable Region

| | |
|---|---|
| QVHLQQSGAELAKPGASVKMSCKASGYTFTSYWIHWIKQRP | SEQ ID |
| GOGLEWIGYINPSSGYHEYNQKFIDKATLTADRSSSTAYMHL | NO: 21 |
| TSLTSEDSAVYYCARSGWLIHGDYYFDFWGQGTTLTVSS | |

Another anti-human PD-L1 mAb that has been reported to be useful for TIC detection of PD-L1 expression in FFPE tissue sections (Chen, B. J. et al., Clin Cancer Res 19: 3462-3473 (2013)) is a rabbit anti-human PD-L1 mAb publicly available from Sino Biological, Inc. (Beijing, P. R. China; Catalog number 10084-R015).

“Framework region” or “FR” as used herein means the immunoglobulin variable regions excluding the CDR regions.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Hypervariable region” refers to the amino acid residues of an antibody that are responsible for antigen-binding. The hypervariable region comprises amino acid residues from a “complementarity determining region” or “CDR” (i.e. LC-CDR1, LC-CDR2 and LC-CDR3 in the light chain variable domain and HC-CDR1, HC-CDR2 and HC-CDR3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues refers to those variable domain residues other than the hypervariable region residues defined herein as CDR residues.

“Immunogenic agent” refers to a composition capable of inducing a humoral and/or cell-mediated immune response. Immunogenic agents may include, for example, attenuated cancerous cells, tumor antigens, antigen presenting cells such as dendritic cells pulsed with tumor derived antigen or nucleic acids, immune stimulating cytokines (e.g., IL-2, IFNα2, GM-CSF), and cells transfected with genes encoding immune stimulating cytokines, such as but not limited to GM-CSF.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat,” as used herein, means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Microsatellite instability (MSI)” refers to the form of genomic instability associated with defective DNA mismatch repair in tumors. See Boland et al., Cancer Research 58, 5258-5257, 1998. In one embodiment, MSI analysis can be carried out using the five National Cancer Institute (NCI) recommended microsatellite markers: BAT25 (GenBank accession no. 9834508), BAT26 (GenBank accession no. 9834505), D5S346 (GenBank accession no. 181171), D2S123 (GenBank accession no. 187953), D17S250 (GenBank accession no. 177030). Additional markers for example, BAT40, BAT34C4, TGF-β-RII and ACTC can be used. Commercially available kits for MSI analysis include, for example, the Promega MSI multiplex PCR assay, FoundationOne® CDx (F1CDx) next generation sequencing based in vitro diagnostic device using DNA isolated from formalin-fixed, paraffin-embedded (FFPE) tumor tissue specimens.

“High frequency microsatellite instability” or “microsatellite instability-high (MSI-H)” refers to a tumor in which two or more of the five NCI markers indicated above show instability in its DNA or ≥30-40% of the total markers in its DNA demonstrate instability (i.e. have insertion/deletion mutations).

“Non-MSI-H cancer” as used herein refers to microsatellite stable (MSS) and low frequency MSI (MSI-L) cancer.

“Microsatellite Stable (MSS)” refers to a tumor in which none of the five NCI markers indicated above show instability in its DNA (i.e. have insertion/deletion mutations).

“Patient” (alternatively referred to as “subject” or “individual” herein) refers to a mammal (e.g., rat, mouse, dog, cat, rabbit) capable of being treated with the methods and compositions of the invention, most preferably a human. In some embodiments, the patient is an adult patient. In other embodiments, the patient is a pediatric patient.

“PD-L1” or “PD-L2” expression means any detectable level of expression of the designated PD-L protein on the cell surface or of the designated PD-L mRNA within a cell or tissue, unless otherwise defined. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR and real-time quantitative RT-PCR.

Several approaches have been described for quantifying PD-L1 protein expression in IHC assays of tumor tissue sections. See, e.g., Thompson et al., PNAS 101 (49): 17174-17179 (2004); Thompson et al., Cancer Res. 66:3381-3385 (2006); Gadiot et al., Cancer 117:2192-2201 (2011); Taube et al., Sci Transl Med 4, 127ra37 (2012); and Toplian et al., New Eng. J Med. 366 (26): 2443-2454 (2012).

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression if at least 1%, and preferably 5% of total tumor cells exhibit histologic evidence of cell-surface membrane staining.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is ≥5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

A tissue section from a tumor that has been stained by IHC with a diagnostic PD-L1 antibody may also be scored for PD-L1 protein expression by assessing PD-L1 expression in both the tumor cells and infiltrating immune cells in the tissue section using a scoring process. See WO 2014/165422. One PD-L1 scoring process comprises examining each tumor nest in the tissue section for staining, and assigning to the tissue section one or both of a modified H score (MHS) and a modified proportion score (MPS). To assign the MHS, four separate percentages are estimated across all of the viable tumor cells and stained mononuclear inflammatory cells in all of the examined tumor nests: (a) cells that have no staining (intensity=0), (b) weak staining (intensity=1+), (c) moderate staining (intensity=2+) and (d) strong staining (intensity=3+). A cell must have at least partial membrane staining to be included in the weak, moderate or strong staining percentages. The estimated percentages, the sum of which is 100%, are then input into the formula of 1×(percent of weak staining cells)+2×(percent of moderate staining cells)+3×(percent of strong staining cells), and the result is assigned to the tissue section as the MHS. The MPS is assigned by estimating, across all of the viable tumor cells and stained mononuclear inflammatory cells in all of the examined tumor nests, the percentage of cells that have at least partial membrane staining of any intensity, and the resulting percentage is assigned to the tissue section as the MPS. In some embodiments, the tumor is designated as positive for PD-L1 expression if the MHS or the MPS is positive.

Another method for scoring/quantifying PD-L1 expression in a tumor is the “combined positive score” or “CPS,” which refers to an algorithm for determining a PD-L1 expression score from a tumor sample of a patient. The CPS is useful in selecting patients for treatment with particular treatment regimens including methods of treatment comprising administration of an anti-PD-1 antibody in which expression of PD-L1 is associated with a higher response rate in a particular patient population relative to same patient population that does not express PD-L1. The CPS is determined by determining the number of viable PD-L1 positive tumor cells, the number of viable PD-L1 negative tumor cells, and the number of viable PD-L1 positive mononuclear inflammatory cells (MIC) in a tumor tissue from a patient having a tumor and calculating the CPS using the following formula:

$$\frac{( \# PD - L 1 positive tumor cells ) + ( \# PD - L 1 positive MIC )}{( \# PD - L 1 positive tumor cells ) + ( PD - L 1 negative tumor cells ) .} \times 100 \%$$

In particular embodiments, the PD-L1 expression scoring method used is the “lymphoma proportion score.” Lymphoma is characterized by a homogeneous population of confluent cells which efface the architecture of the lymph node or the architecture of metastatic site. The “LPS” or “lymphoma proportion score” is the percentage of this population of cells which express PD-L1. When determining the LPS, no attempt is made to distinguish the truly neoplastic cells from the reactive cells. PD-L1 expression is characterized by partial or complete membrane staining at any intensity.

Yet another scoring method for PD-L1 expression is the “TPS” or “tumor proportion score,” which is the percentage of tumor cells expressing PD-L1 on the cell membrane. TPS typically includes the percentage of neoplastic cells expressing PD-L1 at any intensity (weak, moderate, or strong), which can be determined using an immunohistochemical assay using a diagnostic anti-human PD-L1 mAb, e.g. antibody 20C3 and antibody 22C3, described, supra. Cells are considered to express PD-L1 if membrane staining is present, including cells with partial membrane staining.

The level of PD-L mRNA expression may be compared to the mRNA expression levels of one or more reference genes that are frequently used in quantitative RT-PCR, such as ubiquitin C.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In some embodiments, PD-L1 expression in a tumor sample is determined to be elevated if PD-L1 protein (and/or PD-L1 mRNA) in the sample is at least 10%, 20%, or 30% greater than in the control.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 3. Pembrolizumab has been approved by the U.S. FDA as described in the Prescribing Information for KEYTRUDA™ (Merck & Co., Inc., Whitehouse Station, NJ USA; initial U.S. approval 2014, updated March 2021).

“Pembrolizumab variant” as used herein means a monoclonal antibody that comprises heavy chain and light chain sequences that are identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g. the variant positions are located in the FR regions or the constant region, and optionally has a deletion of the C-terminal lysine residues of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“Pharmaceutical formulation” refers to preparations which are in such form as to permit the active ingredients to be effective, and which contains no additional components which are toxic to the subjects to which the formulation would be administered.

“Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

Pharmacokinetic “steady state” is a period of time during which any accumulation of drug concentrations owing to multiple doses has been maximized and systemic drug exposure is considered uniform after each subsequent dose administered; in the specific case of pembrolizumab, steady state is achieved at and after ˜16 weeks of administration.

“Platinum-containing chemotherapy” (also known as platins) refers to the use of chemotherapeutic agent(s) used to treat cancer that are coordination complexes of platinum. Platinum-containing chemotherapeutic agents are alkylating agents that crosslink DNA, resulting in ineffective DNA mismatch repair and generally leading to apoptosis. Examples of platins include cisplatin, carboplatin, and oxaliplatin.

“RECIST 1.1 Response Criteria” as used herein means the definitions set forth in Eisenhauer, E. A. et al., Eur. J. Cancer 45:228-247 (2009) for target lesions or non-target lesions, as appropriate based on the context in which response is being measured.

“Therapeutic agent” refers to an additional agent relative to the anti-PD-1 antibody or antigen-binding fragment thereof. A therapeutic agent may be, e.g., a chemotherapeutic, a biotherapeutic agent, or an immunogenic agent.

“Tissue section” refers to a single part or piece of a tissue sample, e.g., a thin slice of tissue cut from a sample of a normal tissue or of a tumor.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor Mutational Burden” or “TMB” as used herein refers to the number of somatic mutations in a tumor's genome and/or the number of somatic mutations per area of the tumor's genome. TMB high (or TMB-H) refers to a tumor with a high mutational burden. In specific embodiments, a tumor is said to be TMB-H if it contains ≥10 mutations/megabase (Mut/Mb). An FDA approved test, such as FoundationOne® CDx is available for solid tumors to determine whether the solid tumor is TMB-H (i.e., has ≥10 mutations/megabase).

“Variable regions” or “V region” as used herein means the segment of IgG chains which is variable in sequence between different antibodies. It extends to Kabat residue 109 in the light chain and 113 in the heavy chain.

### II. PD-1 ANTIBODIES AND ANTIGEN BINDING FRAGMENTS USEFUL IN THE INVENTION

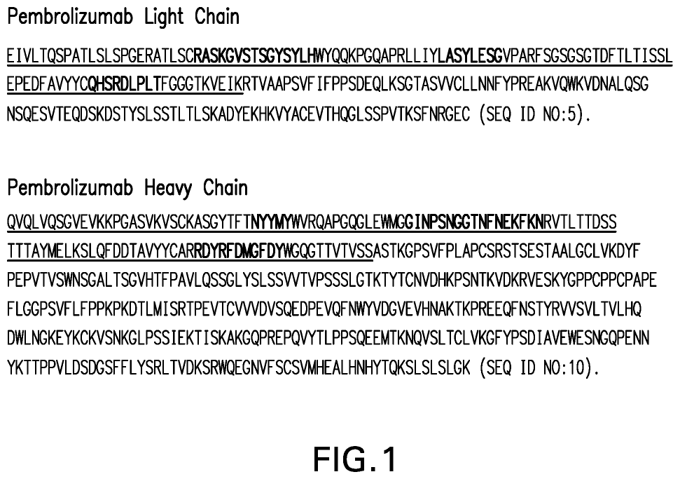

Examples of mAbs that bind to human PD-1, useful in the formulations, treatment methods, compositions, and uses of the invention, are described in U.S. Pat. Nos. 7,521,051, 8,008,449, and 8,354,509. Specific anti-human PD-1 mAbs useful as the PD-1 antagonist in the treatment methods, compositions, and uses of the invention include: pembrolizumab (formerly known as MK-3475, SCH 900475 and lambrolizumab), a humanized IgG4 mAb with the structure described in WHO Drug Information, Vol. 27, No. 2, pages 161-162 (2013) and which comprises the heavy and light chain amino acid sequences shown in FIG. 1, and the humanized antibodies h409A11, h409A16 and h409A17, which are described in WO 2008/156712 and in Table 3.

In some embodiments of the treatment methods, compositions, kits and uses of the invention, the anti-PD-1 antibody, or antigen binding fragment thereof, comprises: (a) light chain CDRs LC-CDR1, LC-CDR2 and LC-CDR3 comprising a sequence of amino acids as set forth in SEQ ID NOs: 1, 2 and 3, respectively, and heavy chain CDRs HC-CDR1, HC-CDR2 and HC-CDR3 comprising a sequence of amino acids as set forth in SEQ ID NOs: 6, 7 and 8, respectively; or (b) light chain CDRs LC-CDR1, LC-CDR2 and LC-CDR3 comprising a sequence of amino acids as set forth in SEQ ID NOs: 11, 12 and 13, respectively, and heavy chain CDRs HC-CDR1, HC-CDR2 and HC-CDR3 comprising a sequence of amino acids as set forth in SEQ ID NOs: 14, 15 and 16, respectively. In some embodiments of the invention, the anti-PD-1 antibody, or antigen binding fragment thereof, is a human antibody. In other embodiments, the anti-PD-1 antibody, or antigen binding fragment thereof, is a humanized antibody. In other embodiments, the anti-PD-1 antibody, or antigen binding fragment thereof, is a chimeric antibody. In specific embodiments, the anti-PD-1 antibody, or antigen binding fragment thereof, is a monoclonal antibody.

In other embodiments of the treatment methods, compositions, kits and uses of the invention, the anti-PD-1 antibody, or antigen binding fragment thereof, specifically binds to human PD-1 and comprises (a) a heavy chain variable region comprising an amino acid sequence as set forth in SEQ ID NO: 9, or a variant thereof, and (b) a light chain variable region comprising an amino acid sequence selected from the group consisting of SEQ ID NO: 4 or a variant thereof, SEQ ID NO: 22 or a variant thereof, and SEQ ID NO: 23 or a variant thereof.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitution in the framework region.

In another embodiment of the treatment methods, compositions, kits and uses of the invention, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO: 1, or a variant thereof, and (b) a light chain comprising or consisting of a sequence of amino acids as set forth in SEQ ID NO: 5, or a variant thereof, SEQ ID NO: 24, or a variant thereof, or SEQ ID NO: 25, or a variant thereof.

In yet another embodiment of the treatment methods, compositions, kits and uses of the invention, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO: 10 and (b) a light chain comprising or consisting of a sequence of amino acids as set forth in SEQ ID NO: 5.

Table 3 below provides a list of the amino acid sequences of exemplary anti-PD-1 mAbs for use in the treatment methods, compositions, kits and uses of the invention.

TABLE 3
Exemplary anti-human PD-1 antibodies
A. Comprises light and heavy chain CDRs of hPD-1.09A in
WO2008/156712 (light and heavy chain CDRs of pembrolizumab)

| | |
|---|---|
| CDRL1 | RASKGVSTSGYSYLH |
| (LC-CDR1) | SEQ ID NO: 1 |
| CDRL2 | LASYLES |
| (LC-CDR2) | SEQ ID NO: 2 |
| CDRL3 | QHSRDLPLT |
| (LC-CDR3) | SEQ ID NO: 3 |
| CDRH1 | NYYMY |
| (HC-CDR1) | SEQ ID NO: 6 |
| CDRH2 | GINPSNGGTNFNEKFKN |
| (HC-CDR2) | SEQ ID NO: 7 |
| CDRH3 | RDYRFDMGFDY |
| (HC-CDR3) | SEQ ID NO: 8 |

B. Comprises light and heavy chain CDRs of hPD-1.08A in WO2008/156712

| | |
|---|---|
| CDRL1 | RASKSVSTSGFSYLH |
| (LC-CDR1) | SEQ ID NO: 11 |
| CDRL2 | LASNLES |
| (LC-CDR2) | SEQ ID NO: 12 |
| CDRL3 | QHSWELPLT |
| (LC-CDR3) | SEQ ID NO: 13 |
| CDRH1 | SYYLY |
| (HC-CDR1) | SEQ ID NO: 14 |
| CDRH2 | GVNPSNGGTNFSEKFKS |
| (HC-CDR2) | SEQ ID NO: 15 |
| CDRH3 | RDSNYDGGFDY |
| (HC-CDR3) | SEQ ID NO: 16 |

C. Comprises the mature h109A heavy chain variable region (VH) and one
of the mature K09A light chain variable (VL) regions in WO 2008/156712

| | |
|---|---|
| Heavy chain VH | QVQLVQSGVEVKKPGASVKVSCKASGYTFTNYYMYWV |
| | RQAPGQGLEWMGGINPSNGGTNFNEKFKNRVTLTTDSST |
| | TTAYMELKSLQFDDTAVYYCARRDYRFDMGFDYWGQG |
| | TTVTVSS |
| | SEQ ID NO: 9 (VH of pembrolizumab) |
| | EIVLTQSPATLSLSPGERATLSCRASKGVSTSGYSYLHWY |
| | QQKPGQAPRLLIYLASYLESGVPARFSGSGSGTDFTLTISS |
| | LEPEDFAVYYCQHSRDLPLTFGGGTKVEIK |
| | SEQ ID NO: 4 (VL of pembrolizumab) or |
| Light chain VL | EIVLTQSPLSLPVTPGEPASISCRASKGVSTSGYSYLHWYL |
| | QKPGQSPQLLIYLASYLESGVPDRFSGSGSGTDFTLKISRV |
| | EAEDVG VYYCQHSRDLPLTFGQGTKLEIK |
| | SEQ ID NO: 22 or |
| | DIVMTQTPLSLPVTPGEPASISCRASKGVSTSGYSYLHWY |
| | LQKPGQSPQLLIYLASYLESGVPDRFSGSGSGTAFTLKISR |
| | VEAEDV GLYYCQHSRDLPLTFGQGTKLEIK |
| | SEQ ID NO: 23 |

D. Comprises the mature 409 heavy chain and one of the mature
K09A light chains in WO 2008/156712

| | |
|---|---|
| Heavy chain | QVQLVQSGVEVKKPGASVKVSCKASGYTFTNYYMYWV |
| | RQAPGQGLEWMGGINPSNGGTNFNEKFKNRVTLTTDSST |
| | TTAYMELKSLQFDDTAVYYCARRDYRFDMGFDYWGQG |
| | TTVTVSSASTKGPSVFPLAPCSRSTSESTAALGCLVKDYFP |
| | EPVTVSWNSGALTSGVHTFPAVLQSSGLYSLSSVVTVPSS |
| | SLGTKTYTCNVDHKPSNTKVDKRVESKYGPPCPPCPAPEF |
| | LGGPSVFLFPPKPKDTLMISRTPEVTCVVVDVSQEDPEVQ |
| | FNWYVDGVEVHNAKTKPREEQFNSTYRVVSVLTVLHQD |
| | WLNGKEYKCKVSNKGLPSSIEKTISKAKGQPREPQVYTLP |
| | PSQEEMTKNQVSLTCLVKGFYPSDIAVEWESNGQPENNY |
| | KTTPPVLDSDGSFFLYSRLTVDKSRWQEGNVFSCSVMHE |
| | ALHNHYTQKSLSLSLGK |
| | SEQ ID NO: 10 (heavy chain of pembrolizumab) |
| Light chain | EIVLTQSPATLSLSPGERATLSCRASKGVSTSGYSYLHWY |
| | QQKPGQAPRLLIYLASYLESGVPARFSGSGSGTDFTLTISS |
| | LEPEDFAVYYCQHSRDLPLTFGGGTKVEIKRTVAAPSVFI |
| | FPPSDEQLKSGTASVVCLLNNFYPREAKVQWKVDNALQS |
| | GNSQESVTEQDSKDSTYSLSSTLTLSKADYEKHKVYACE |
| | VTHQGLSSPVTKSFNRGEC |
| | SEQ ID NO: 5 (light chain of pembrolizumab) or |
| | EIVLTQSPLSLPVTPGEPASISCRASKGVSTSGYSYLHWYL |
| | QKPGQSPQLLIYLASYLESGVPDRFSGSGSGTDFTLKISRV |
| | EAEDVGVYYCQHSRDLPLTFGQGTKLEIKRTVAAPSVFIF |
| | PPSDEQLKSGTASVVCLLNNFYPREAKVQWKVDNALQS |
| | GNSQESVTEQDSKDSTYSLSSTLTLSKADYEKHKVYACE |
| | VTHQGLSSPVTKSFNRGEC |
| | SEQ ID NO: 24 or |
| | DIVMTQTPLSLPVTPGEPASISCRASKGVSTSGYSYLHWY |
| | LQKPGQSPQLLIYLASYLESGVPDRFSGSGSGTAFTLKISR |
| | VEAEDVGLYYCQHSRDLPLTFGQGTKLEIKRTVAAPSVFI |
| | FPPSDEQLKSGTASVVCLLNNFYPREAKVQWKVDNALQS |
| | GNSQESVTEQDSKDSTYSLSSTLTLSKADYEKHKVYACE |
| | VTHQGLSSPVTKSFNRGEC |
| | SEQ ID NO: 25 |

### III. METHODS AND USES OF THE INVENTION

The invention provides a method of treating cancer in a human patient comprising subcutaneously administering to the patient about 280 mg to about 450 mg of an anti-PD-1 antibody, or antigen-binding fragment thereof, once approximately every three weeks, wherein the anti-PD-1 antibody, or antigen binding fragment thereof, comprises: (a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively; or (b) light chain CDRs LC-CDR1, LC-CDR2 and LC-CDR3 comprising a sequence of amino acids as set forth in SEQ ID NOs: 11, 12 and 13, respectively, and heavy chain CDRs HC-CDR1, HC-CDR2 and HC-CDR3 comprising a sequence of amino acids as set forth in SEQ ID NOs: 14, 15 and 16, respectively. In particular embodiments, the anti-PD-1 antibody, or antigen binding fragment thereof, is administered every three weeks. In particular embodiments of the invention, the anti-PD-1 antibody, or antigen-binding fragment thereof, is pembrolizumab. In other embodiments, the anti-PD-1 antibody, or antigen-binding fragment thereof, is a pembrolizumab variant.

The invention provides a method of treating cancer in a human patient comprising subcutaneously administering an anti-PD-1 antibody, or antigen binding fragment thereof, to the patient at a dose that is at least 1.6 times higher than a different, therapeutically effective dose administered by an IV route of administration, wherein the subcutaneous dose is administered at the same frequency as the frequency of the different, therapeutically effective dose, wherein the anti-PD-1 antibody, or antigen binding fragment thereof, comprises: (a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively; or (b) light chain CDRs LC-CDR1, LC-CDR2 and LC-CDR3 comprising a sequence of amino acids as set forth in SEQ ID NOs: 11, 12 and 13, respectively, and heavy chain CDRs HC-CDR1, HC-CDR2 and HC-CDR3 comprising a sequence of amino acids as set forth in SEQ ID NOs: 14, 15 and 16, respectively. In particular embodiments, the anti-PD-1 antibody, or antigen binding fragment thereof, is administered every three weeks. In particular embodiments of the invention, the anti-PD-1 antibody, or antigen-binding fragment thereof, is pembrolizumab. In other embodiments, the anti-PD-1 antibody, or antigen-binding fragment thereof, is a pembrolizumab variant.

The invention provides a method of treating cancer in a human patient comprising subcutaneously administering approximately every three weeks an anti-PD-1 antibody, or antigen-binding fragment thereof, to the patient at a dose that is at least 1.6 times higher than a 200 mg or a 2 mg/kg dose of the anti-PD-1 antibody, or antigen-binding fragment thereof, administered by an IV route of administration approximately every three weeks, wherein the anti-PD-1 antibody, or antigen binding fragment thereof, comprises: (a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively; or (b) light chain CDRs LC-CDR1, LC-CDR2 and LC-CDR3 comprising a sequence of amino acids as set forth in SEQ ID NOs: 11, 12 and 13, respectively, and heavy chain CDRs HC-CDR1, HC-CDR2 and HC-CDR3 comprising a sequence of amino acids as set forth in SEQ ID NOs: 14, 15 and 16, respectively. In particular embodiments, the anti-PD-1 antibody, or antigen binding fragment thereof, is administered every three weeks. In particular embodiments, In particular embodiments of the invention, the anti-PD-1 antibody, or antigen-binding fragment thereof, is pembrolizumab. In other embodiments, the anti-PD-1 antibody, or antigen-binding fragment thereof, is a pembrolizumab variant.

The invention provides a method of treating cancer in a human patient comprising subcutaneously administering approximately every three weeks an anti-PD-1 antibody, or antigen-binding fragment thereof, to the patient at a dose that is at least 1.6 times higher than a 200 mg or a 2 mg/kg dose of the anti-PD-1 antibody, or antigen-binding fragment thereof, wherein the anti-PD-1 antibody, or antigen binding fragment thereof, comprises: (a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively; or (b) light chain CDRs LC-CDR1, LC-CDR2 and LC-CDR3 comprising a sequence of amino acids as set forth in SEQ ID NOs: 11, 12 and 13, respectively, and heavy chain CDRs HC-CDR1, HC-CDR2 and HC-CDR3 comprising a sequence of amino acids as set forth in SEQ ID NOs: 14, 15 and 16, respectively. In particular embodiments, the anti-PD-1 antibody, or antigen binding fragment thereof, is administered every three weeks. In particular embodiments of the invention, the anti-PD-1 antibody, or antigen-binding fragment thereof, is pembrolizumab. In other embodiments, the anti-PD-1 antibody, or antigen-binding fragment thereof, is a pembrolizumab variant.

In some embodiments, the dose is at least 1.65, 1.7, 1.75, 1.8, 1.85, 1.9, 1.95, 2.0, 2.05, or 2.1 times higher than a 200 mg of a 2 mg/kg dose of the anti-PD-1 antibody, or antigen binding fragment thereof.

In some embodiments, the dose is at least 1.65 times higher than a 200 mg or a 2 mg/kg dose of the anti-PD-1 antibody or antigen binding fragment thereof. In some embodiments, the dose is at least 1.7 times higher than a 200 mg or a 2 mg/kg dose of the anti-PD-1 antibody or antigen binding fragment thereof. In some embodiments, the dose is at least 1.75 times higher than a 200 mg or a 2 mg/kg dose of the anti-PD-1 antibody or antigen binding fragment thereof. In some embodiments, the dose is at least 1.8 times higher than a 200 mg or a 2 mg/kg dose of the anti-PD-1 antibody or antigen binding fragment thereof. In some embodiments, the dose is at least 1.85 times higher than a 200 mg or a 2 mg/kg dose of the anti-PD-1 antibody or antigen binding fragment thereof. In some embodiments, the dose is at least 1.9 times higher than a 200 mg or a 2 mg/kg dose of the anti-PD-1 antibody or antigen binding fragment thereof. In some embodiments, the dose is at least 1.95 times higher than a 200 mg or a 2 mg/kg dose of the anti-PD-1 antibody or antigen binding fragment thereof. In some embodiments, the dose is at least 2.0 times higher than a 200 mg or a 2 mg/kg dose of the anti-PD-1 antibody or antigen binding fragment thereof. In some embodiments, the dose is at least 2.05 times higher than a 200 mg or a 2 mg/kg dose of the anti-PD-1 antibody or antigen binding fragment thereof. In some embodiments, the dose is at least 2.1 times higher than a 200 mg or a 2 mg/kg dose of the anti-PD-1 antibody or antigen binding fragment thereof. In particular embodiments, the dose is at least 1.875 times higher than a 200 mg or a 2 mg/kg dose of the anti-PD-1 antibody or antigen binding fragment thereof. In other particular embodiments, the dose is at least 1.9 times higher than a 200 mg or a 2 mg/kg dose of the anti-PD-1 antibody or antigen binding fragment thereof.

In some embodiments, the dose is 1.6 to 2.1, 1.7-2.1, 1.8-2.1, 1.9 to 2.1 times higher than a 200 mg or a 2 mg/kg dose of the anti-PD-1 antibody, or antigen binding fragment thereof. In some embodiments, the dose is at 1.6 to 2.1 times higher than a 200 mg or a 2 mg/kg dose of the anti-PD-1 antibody or antigen binding fragment thereof. In some embodiments, the dose is 1.7 to 2.1 times higher than a 200 mg or a 2 mg/kg dose of the anti-PD-1 antibody or antigen binding fragment thereof. In some embodiments, the dose is 1.8 to 2.1 times higher than a 200 mg or a 2 mg/kg dose of the anti-PD-1 antibody or antigen binding fragment thereof. In some embodiments, the dose is 1.875 to 2.1 times higher than a 200 mg or a 2 mg/kg dose of the anti-PD-1 antibody or antigen binding fragment thereof. In some embodiments, the dose is 1.9 to 2.1 times higher than a 200 mg or a 2 mg/kg dose of the anti-PD-1 antibody or antigen binding fragment thereof.

In embodiments of any of the methods of the invention, the bioavailability of the anti-PD-1 antibody, or antigen binding fragment thereof, is at least 63%. In embodiments of any of the methods described above, the bioavailability of the anti-PD-1 antibody, or antigen binding fragment thereof, is at least 64%. In embodiments of any of the methods described above, the bioavailability of the anti-PD-1 antibody, or antigen binding fragment thereof, is 66%.

In some embodiments of the methods of the invention, the subcutaneous administration of the anti-PD-1 antibody, or antigen binding fragment thereof, (e.g., pembrolizumab) dose results in a C_{trough} that is the same, or greater than, the C_{trough} of the dose administered by an IV route of administration. In an embodiment of any of the methods described above, the subcutaneous administration of the anti-PD-1 antibody, or antigen binding fragment thereof, results in a ratio of subcutaneous C_{trough} to IV C_{trough} of at least 1, at least 1.2, at least 1.3, at least 1.4, at least 1.5, or at least 1.6. In some embodiments, the subcutaneous administration results in a PK profile having a SC:IV C_{trough} ratio of at least 1.0 or greater. In some embodiments, the subcutaneous administration results in a SC:IV C_{trough} ratio of at least 1.2 or greater. In some embodiments, the subcutaneous administration results in a SC:IV C_{trough} ratio of at least 1.3 or greater. In some embodiments, the subcutaneous administration results in a SC:IV C_{trough} ratio of at least 1.4 or greater. In some embodiments, the subcutaneous administration results in a SC:IV C_{trough} ratio of at least 1.5 or greater. In some embodiments, the subcutaneous administration results in a SC:IV C_{trough} ratio of at least 1.6 or greater.

In some embodiments of the methods of the invention, the subcutaneous administration of the anti-PD-1 antibody, or antigen binding fragment thereof, (e.g., pembrolizumab) dose results in a SC:IV C_{trough} ratio of 1.0 to 1.6, 1.1 to 1.6, 1.2 to 1.6, 1.3 to 1.6, 1.4 to 1.6, 1.2 to 1.5, 1.3 to 1.5, 1.4 to 1.5 or 1.3 to 1.4.

In some embodiments of the methods of the invention, the subcutaneous administration of the anti-PD-1 antibody, or antigen binding fragment thereof, (e.g., pembrolizumab) dose results in a SC:IV C_{trough} ratio of 1.0 to 1.6. In some embodiments, the subcutaneous administration results in a SC:IV C_{trough} ratio of 1.1 to 1.6. In some embodiments, the subcutaneous administration results in a SC:IV C_{trough} ratio of 1.2 to 1.6. In some embodiments, the subcutaneous administration results in a SC:IV C_{trough} ratio of 1.3 to 1.6. In some embodiments, the subcutaneous administration results in a SC:IV C_{trough} ratio of 1.4 to 1.6. In some embodiments, the subcutaneous administration results in a SC:IV C_{trough} ratio of at 1.2 to 1.5. In some embodiments, the subcutaneous administration results in a SC:IV C_{trough} ratio of at 1.3 to 1.5. In some embodiments, the subcutaneous administration results in a SC:IV C_{trough} ratio of at 1.4 to 1.5. In some embodiments, the subcutaneous administration results in a SC:IV C_{trough} ratio of at 1.3 to 1.4.

In some embodiments of the methods of the invention, the subcutaneous administration of the anti-PD-1 antibody, or antigen binding fragment thereof, (e.g., pembrolizumab) dose results in a AUC_{(0-3 weeks)} that is greater than the AUC_{(0-3 weeks)} of a 200 mg or 2 mg/kg dose of the anti-PD-1 antibody, or antigen binding fragment thereof, administered by an IV route of administration after six cycles of administration (e.g., after six cycles of once every three week dosing). In some embodiments, the subcutaneous administration results in a PK profile having a SC:IV AUC_{(0-3 weeks)} ratio of at least 1.0 or greater after six cycles of administration.

In some embodiments of the methods of the invention, the parameters above (e.g. AUC_{(0-3 weeks)}, C_{trough}) are compared to those resulting from a dose of the anti-PD1 antibody, or antigen binding fragment thereof, which is administered by an IV route of administration, wherein the dose is 200 mg.

In some embodiments of the methods of the invention, the cancer is selected from the group consisting of: melanoma, lung cancer, head and neck cancer, bladder cancer, breast cancer, gastrointestinal cancer, multiple myeloma, hepatocellular cancer, merkel cell carcinoma, cutaneous squamous cell carcinoma, lymphoma, renal cancer, mesothelioma, ovarian cancer, esophageal cancer, anal cancer, biliary tract cancer, colorectal cancer, endometrial cancer, cervical cancer, thyroid cancer, salivary cancer, prostate cancer (e.g. hormone refractory prostate adenocarcinoma), pancreatic cancer, colon cancer, liver cancer, thyroid cancer, glioblastoma, glioma, and other neoplastic malignancies.

In some embodiments the lung cancer in non-small cell lung cancer.

In alternate embodiments, the lung cancer is small-cell lung cancer.

In some embodiments, the lymphoma is Hodgkin lymphoma.

In other embodiments, the lymphoma is non-Hodgkin lymphoma. In particular embodiments, the lymphoma is primary mediastinal large B-cell lymphoma (PMBCL). In some embodiments, the lymphoma is diffuse large B-cell lymphoma (DLBCL).

In some embodiments, the breast cancer is triple negative breast cancer.

In further embodiments, the breast cancer is ER+/HER2−breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer. In other embodiments, the cancer is squamous cell carcinoma of the head and neck.

In some embodiments, the cancer is metastatic colorectal cancer with high levels of microsatellite instability (MSI-H).

In some embodiments, the cancer is a solid tumor with a high level of microsatellite instability (MSI-H).

In some embodiments, the cancer is a solid tumor with a high mutational burden.

In some embodiments of the methods of the invention, the cancer is selected from the group consisting of: melanoma, non-small cell lung cancer, small cell lung cancer, head and neck squamous cell cancer, classical Hodgkin lymphoma, primary mediastinal large B-cell lymphoma, urothelial carcinoma, microsatellite instability-high or mismatch repair deficient cancer, gastric cancer, esophageal cancer, cervical cancer, hepatocellular carcinoma, Merkel cell carcinoma, renal cell carcinoma, endometrial carcinoma, a cancer characterized by a tumor having a high mutational burden, cutaneous squamous cell carcinoma, and triple negative breast cancer.

In some embodiments of the methods of the invention, the cancer is selected from the group consisting of: melanoma, non-small cell lung cancer, head and neck squamous cell cancer, classical Hodgkin lymphoma, primary mediastinal large B-cell lymphoma, urothelial carcinoma, microsatellite instability-high or mismatch repair deficient cancer, gastric cancer, esophageal cancer, cervical cancer, hepatocellular carcinoma, Merkel cell carcinoma, renal cell carcinoma, endometrial carcinoma, a cancer characterized by a tumor having a high mutational burden, cutaneous squamous cell carcinoma, and triple negative breast cancer.

In some embodiments of methods of the invention, the anti-PD-1 antibody, or antigen binding fragment thereof, is administered to the patient about once every three weeks for 12 weeks or more. In other embodiments, the anti-PD-1 antibody, or antigen binding fragment thereof is administered to the patient about once every three weeks for 18 weeks or more, 24 weeks or more, 30 weeks or more, 36 weeks or more, 42 weeks or more, 48 weeks or more, 54 weeks or more, 60 weeks or more, 66 weeks or more, 72 weeks or more, 78 weeks or more, 84 weeks or more, 90 weeks or more, 96 weeks or more, or 102 weeks or more.

In a first embodiment (Embodiment E1), the invention comprises a method of treating cancer in a human patient comprising subcutaneously administering about 280 mg to about 450 mg of an anti-PD-1 antibody (e.g., pembrolizumab), or antigen binding fragment thereof, to the patient once approximately every three weeks. In specific embodiments, the anti-PD-1 antibody, or antigen binding fragment thereof, is administered once every three weeks.

In a second embodiment (Embodiment E2), the invention comprises a method of treating unresectable or metastatic melanoma in a human patient comprising subcutaneously administering about 280 mg to about 450 mg of an anti-PD-1 antibody (e.g., pembrolizumab), or antigen binding fragment thereof, to the patient once approximately every three weeks. In specific embodiments, the anti-PD-1 antibody, or antigen binding fragment thereof, is administered once every three weeks.

In a third embodiment (Embodiment E3), the invention comprises a method of treating metastatic non-small cell lung cancer (NSCLC) in a human patient comprising subcutaneously administering about 280 mg to about 450 mg of an anti-PD-1 antibody (e.g., pembrolizumab), or antigen binding fragment thereof, to the patient once approximately every three weeks. In specific embodiments, the anti-PD-1 antibody, or antigen binding fragment thereof, is administered once every three weeks.

In a sub-embodiment of Embodiment E3 (Embodiment E3-A), the patient has a tumor with high PD-L1 expression [(Tumor Proportion Score (TPS)≥50%)] and was not previously treated with platinum-containing chemotherapy.

In a further sub-embodiment of Embodiment E3 (Embodiment E3-B), the patient has a tumor with PD-L1 expression (TPS≥1%) and was previously treated with platinum-containing chemotherapy. In specific embodiments of Embodiment E3-B, the patient had disease progression on or after receiving platinum-containing chemotherapy.

In another sub-embodiment of Embodiment E3 (Embodiment E3-C), the patient has a tumor with PD-L1 expression (TPS≥1%) and was not previously treated with platinum-containing chemotherapy.

In yet another sub-embodiment of Embodiment E3 (Embodiment E3-D), the patient's tumor is not tested for PD-L1 expression. In this embodiment, the patient is treated with the anti-PD-1 antibody, or antigen binding fragment thereof, regardless of PD-L1 expression. In specific embodiments, the patient was not previously treated with platinum-containing chemotherapy.

In certain embodiments of Embodiment E3 (including Embodiments E3-A, E3-B, and E3-C), the PD-L1 TPS is determined by an FDA-approved test.

In certain embodiments of Embodiment E3 (including Embodiments E3-A, E3-B, E3-C and E3-D), the patient's tumor has no EGFR or ALK genomic aberrations.

In certain embodiments of Embodiment E3 (including Embodiments E3-A, E3-B, E3-C and E3-D), the patient's tumor has an EGFR or ALK genomic aberration and had disease progression on or after receiving treatment for the EGFR or ALK aberration(s) prior to receiving the anti-PD-1 antibody, or antigen binding fragment thereof.

In a fourth embodiment (Embodiment E4), the invention comprises a method of treating metastatic non-small cell lung cancer (NSCLC) in a human patient comprising: (1) subcutaneously administering about 280 mg to about 450 mg of an anti-PD-1 antibody (e.g., pembrolizumab), or antigen binding fragment thereof, to the patient once every approximately three weeks, and (2) administering pemetrexed and platinum chemotherapy (e.g., carboplatin) to the patient. In sub-embodiments of Embodiment E4, the patient was not previously treated with an anti-cancer therapeutic prior to starting the combination treatment regimen with the anti-PD-1 antibody, or antigen binding fragment thereof, pemetrexed and carboplatin.

In certain embodiments of Embodiments E3 and E4 (including sub-embodiments thereof), the patient has nonsquamous non-small cell lung cancer.

In certain embodiments of Embodiments E3 and E4 (including sub-embodiments thereof) the patient is also treated with carboplatin and paclitaxel or nab-paclitaxel.

In sub-embodiments of Embodiment E4, pemetrexed is administered to the patient in an amount of 500 mg/m².

In sub-embodiments of Embodiment E4, pemetrexed is administered to the patient in an amount of 500 mg/m² every 3 weeks.

In sub-embodiments of Embodiment E4, pemetrexed is administered to the patient via intravenous infusion every 21 days. In specific embodiments, the infusion time is about 10 minutes.

In sub-embodiments of Embodiment E4 (Embodiment E4-A), the invention further comprises administering about 400 μg to about 1000 μg of folic acid to the patient once per day, beginning about 7 days prior to administering pemetrexed to the patient and continuing until about 21 days after the patient is administered the last dose of pemetrexed. In certain embodiments the folic acid is administered orally.

In sub-embodiments of Embodiments E4 and E4-A (Embodiment E4-B), the invention further comprises administering about 1 mg of vitamin B₁₂ to the patient about 1 week prior to the first administration of pemetrexed and about every three cycles of pemetrexed administration (i.e., approximately every 9 weeks). In certain embodiments, the vitamin B₁₂ is administered intramuscularly.

In sub-embodiments of Embodiments E4, E4-A and E4-B (Embodiment E4-C), the invention further comprises administering about 4 mg of dexamethasone to the patient twice a day on the day before, the day of, and the day after pemetrexed administration. In certain embodiments the dexamethasone is administered orally.

In a fifth embodiment (Embodiment E5), the invention comprises a method of treating recurrent or metastatic head and neck squamous cell cancer (HNSCC) in a human patient comprising subcutaneously administering about 280 mg to about 450 mg of an anti-PD-1 antibody (e.g., pembrolizumab), or antigen binding fragment thereof, to the patient once approximately every three weeks.

In sub-embodiments of Embodiment E5 (Embodiment E5-A), the patient was previously treated with platinum-containing chemotherapy. In certain embodiments, the patient had disease progression on or after platinum-containing chemotherapy.

In sub-embodiments of Embodiment E5 (Embodiment E5-B), the patient has metastatic or unresectable, recurrent HNSCC and the method further comprises administering platinum and 5-FU (Fluorouracil) for first-line treatment of the HNSCC.

In sub-embodiments of Embodiments E5 (Embodiment E5-C), the anti-PD-1 antibody (e.g., pembrolizumab) is administered as a single agent for the first line treatment of a patient with metastatic or unresectable, recurrent HNSCC, wherein the patient's tumors express PD-L1 (CPS≥1%).

In a sixth embodiment (Embodiment E6), the invention comprises a method of treating refractory classical Hodgkin lymphoma (cHL) in a human patient comprising subcutaneously administering about 280 mg to about 450 mg of an anti-PD-1 antibody (e.g., pembrolizumab), or antigen binding fragment thereof, to the patient once approximately every three weeks.

In a seventh embodiment (Embodiment E7), the invention comprises a method of treating classical Hodgkin lymphoma (cHL) in a human patient comprising subcutaneously administering about 280 mg to about 450 mg of an anti-PD-1 antibody (e.g., pembrolizumab), or antigen binding fragment thereof, to the patient once every approximately three weeks, wherein the patient has relapsed after (a) one or more lines of therapy for cHL, (b) 2 or more lines of therapy for cHL, or (c) 3 or more lines of therapy for cHL.

In sub-embodiments of Embodiments E6 and E7, the patient is an adult patient.

In alternative sub-embodiments of Embodiments E6 and E7, the patient is a pediatric patient.

In an eighth embodiment (Embodiment E8), the invention comprises a method of treating locally advanced or metastatic urothelial carcinoma in a human patient comprising subcutaneously administering about 280 mg to about 450 mg of an anti-PD-1 antibody (e.g., pembrolizumab), or antigen binding fragment thereof, to the patient once approximately every three weeks.

In sub-embodiments of Embodiment E8, the patient is not eligible for cisplatin-containing chemotherapy.

In sub-embodiments of Embodiment E8, the patient had disease progression during or following platinum-containing chemotherapy or within 12 months of neoadjuvant or adjuvant treatment with platinum-containing chemotherapy.

In sub-embodiments of Embodiment E8, the patient's tumor expresses PD-L1. In other sub-embodiments of Embodiment E8, the patient tumor expresses PD-L1 (CPS≥10).

In a ninth embodiment (Embodiment E9), the invention comprises a method of treating unresectable or metastatic, microsatellite instability-high (MSI-H) or mismatch repair (MMR) deficient solid tumors in a human patient comprising subcutaneously administering 280 mg to about 450 mg of an anti-PD-1 antibody (e.g., pembrolizumab), or antigen binding fragment thereof, to the patient once approximately every three weeks.

In a sub-embodiment of Embodiment E9, the patient had disease progression following prior anti-cancer treatment.

In a tenth embodiment (Embodiment E10), the invention comprises a method of treating unresectable or metastatic, MSI-H or MMR deficient colorectal cancer in a human patient comprising subcutaneously administering about 280 mg to about 450 mg of an anti-PD-1 antibody (e.g., pembrolizumab), or antigen binding fragment thereof, to the patient once approximately every three weeks.

In a sub-embodiment of Embodiment E10, the patient had disease progression following prior treatment with a fluoropyrimidine, oxaliplatin, and irinotecan.

In an eleventh embodiment (Embodiment E11), the invention comprises a method of treating recurrent locally advanced or metastatic gastric cancer in a human patient comprising subcutaneously administering about 280 mg to about 450 mg of an anti-PD-1 antibody (e.g., pembrolizumab), or antigen binding fragment thereof, to the patient once approximately every three weeks. In specific embodiments, the invention further comprises treating the patient with trastuzumab, fluoropyrimidine and platinum-containing chemotherapy. In specific embodiments, the treatment with the anti-PD-1 antibody, trastuzumab, fluoropyrimidine and platinum-containing chemotherapy is a first-line treatment.

In a twelfth embodiment (Embodiment E12), the invention comprises a method of treating recurrent locally advanced or metastatic gastroesophageal junction adenocarcinoma in a human patient comprising subcutaneously administering about 280 mg to about 450 mg an anti-PD-1 antibody (e.g., pembrolizumab), or antigen binding fragment thereof, to the patient once approximately every three weeks.

In sub-embodiments of Embodiments E11 and E12, the patient's tumor expresses PD-L1. In sub-embodiments of Embodiments E11 and E12, the patient's tumor has a PD-L1 Combined Positive Score (CPS)≥1.

In sub-embodiments of Embodiments E11 and E12, the patient had disease progression on or after one or more prior lines of therapy. In specific embodiments, the prior lines of therapy include fluoropyrimidine and platinum-containing chemotherapy.

In sub-embodiments of Embodiments E11 and E12, the patient had disease progression on or after two or more prior lines of therapy including fluoropyrimidine- and platinum-containing chemotherapy.

In sub-embodiments of Embodiments E11 and E12, the patient had disease progression on or after one or more prior lines of therapy including HER2/neu-targeted therapy.

In sub-embodiments of Embodiments E11 and E12, the patient had disease progression on or after two or more prior lines of therapy including HER2/neu-targeted therapy.

In a thirteenth embodiment (Embodiment E13), the invention comprises a method of treating cancer in a human patient comprising subcutaneously administering about 280 mg to about 450 mg of an anti-PD-1 antibody (e.g. pembrolizumab), or antigen binding fragment thereof, to the patient once every approximately three weeks, wherein the patient has a cancer selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, hepatocellular carcinoma, merkel cell carcinoma renal cell carcinoma, endometrial carcinoma, cutaneous squamous cell carcinoma, thyroid cancer, and salivary cancer.

In a fourteenth embodiment (Embodiment E14), the invention comprises a method of treating cancer in a human patient comprising subcutaneously administering about 280 mg to about 450 mg of an anti-PD-1 antibody (e.g. pembrolizumab), or antigen binding fragment thereof, to the patient once approximately every three weeks, wherein the patient has small-cell lung cancer. In a sub-embodiment, the patient was previously treated with platinum-based chemotherapy and at least one other prior line of therapy.

In a fifteenth embodiment (Embodiment E15), the invention comprises a method of treating non-Hodgkin lymphoma in a human patient comprising subcutaneously administering about 280 mg to about 450 mg of an anti-PD-1 antibody (e.g. pembrolizumab), or antigen binding fragment thereof, to the patient once approximately every three weeks.

In a sub-embodiment of Embodiment E15, the non-Hodgkin lymphoma is primary mediastinal large B-cell lymphoma (PMBCL). In some embodiments where the patient has PMBCL, the patient has refractory PMBCL. In some embodiments, the patient has relapsed after one or more prior lines of therapy. In some embodiments, the patient has relapsed after two or more prior lines of therapy. In some embodiments, the patient was not previously treated with another line of therapy. In some embodiments, the patient is an adult. In some embodiments, the patient is a pediatric patient.

In a sixteenth embodiment (Embodiment E16), the invention comprises a method of treating metastatic squamous NSCLC in a human patient comprising: (1) subcutaneously administering about 280 mg to about 450 mg of an anti-PD-1 antibody (e.g., pembrolizumab), or antigen binding fragment thereof, to the patient once approximately every three weeks, and (2) administering (i) carboplatin and paclitaxel, or (ii) carboplatin and nab-paclitaxel to the patient.

In a seventeenth embodiment (Embodiment E17), the invention comprises a method of treating Merkel cell carcinoma (MCC) in a human patient comprising subcutaneously administering about 280 mg to about 450 mg of an anti-PD-1 antibody (e.g., pembrolizumab), or antigen binding fragment thereof, to the patient once approximately every three weeks. In particular sub-embodiments of Embodiment E17, the cancer is recurrent, locally advanced MCC. In particular sub-embodiments of Embodiment E17, the cancer is metastatic MCC.

In sub-embodiments of Embodiment E17, the patient is an adult patient. In alternative sub-embodiments of Embodiment E17, the patient is a pediatric patient.

In a eighteenth embodiment (Embodiment E18), the invention comprises a method for adjuvant therapy of melanoma in a human patient comprising subcutaneously administering about 280 mg to about 450 mg of an anti-PD-1 antibody (e.g., pembrolizumab), or antigen binding fragment thereof, to a patient once every approximately every about three weeks, wherein the patient has previously had one or more melanoma lesions resected. In sub-embodiments of Embodiment E18, the method comprises treating resected high-risk stage III melanoma. In sub-embodiments of Embodiment E18, the method comprises treating resected stage IIB or IIC melanoma.

In a nineteenth embodiment (Embodiment E19), the invention comprises a method of treating hepatocellular carcinoma (HCC) in a human patient comprising subcutaneously administering about 280 mg to about 450 mg of an anti-PD-1 antibody (e.g., pembrolizumab), or antigen binding fragment thereof, to the patient once approximately every three weeks. In some embodiments of Embodiment E19, the patient was previously treated with sorafenib.

In a twentieth embodiment (Embodiment E20), the invention comprises a method of treating renal cell carcinoma (RCC) in a human patient comprising subcutaneously administering about 280 mg to about 450 mg of an anti-PD-1 antibody (e.g., pembrolizumab), or antigen binding fragment thereof, to the patient once approximately every three weeks.

In sub-embodiments, of Embodiment E20, the cancer is advanced clear cell RCC.

In sub-embodiments of Embodiment E20, the patient has advanced or metastatic renal cell carcinoma (RCC).

In sub-embodiments, of Embodiment E20 (Embodiment E20A), the patient is further treated with axitinib. In sub-embodiments of the invention, axitinib is taken orally.

In particular embodiments of Embodiment E20A, 5 mg axitinib is taken by the patient approximately every 12 hours or twice a day.

In alternative embodiments of Embodiment E20A, the axitinib dosage is 2.5 mg, 3 mg, 7 mg, or 10 mg twice daily.

In sub-embodiments, of Embodiment E20 (Embodiment E20B), the patient is further treated with lenvatinib.

In a twenty-first embodiment (Embodiment E21), the invention comprises a method of treating breast cancer in a human patient comprising subcutaneously administering about 280 mg to about 450 mg of an anti-PD-1 antibody (e.g., pembrolizumab), or antigen binding fragment thereof, to the patient once approximately every three weeks.

In a sub-embodiment of Embodiment E21, the breast cancer is triple negative breast cancer. In a further sub-embodiment, the patient is further treated with chemotherapy. In a further sub-embodiment, the TNBC is recurrent unresectable or metastatic TNBC and the patient's tumors express PD-L1 (CPS≥10).

In a sub-embodiment of Embodiment E21, the breast cancer is ER+/HER2− breast cancer.

In a further sub-embodiment of Embodiment E21, the patient has high-risk early stage TNBC and the method further comprises treating the patient with chemotherapy as neoadjuvant treatment, and then treating the patient with the anti-PD-1 antibody (e.g. pembrolizumab) as a single agent as adjuvant treatment after surgery.

In a twenty-second embodiment (Embodiment E22), the invention comprises a method of treating nasopharyngeal cancer in a human patient comprising subcutaneously administering about 280 mg to about 450 mg of an anti-PD-1 antibody (e.g., pembrolizumab), or antigen binding fragment thereof, to the patient once approximately every three weeks.

In a twenty-third embodiment (Embodiment E23), the invention comprises a method of treating thyroid cancer in a human patient comprising administering 400 mg of an anti-PD-1 antibody (e.g., pembrolizumab), or antigen binding fragment thereof, to the patient once approximately every three weeks.

In a twenty-fourth embodiment (Embodiment E24), the invention comprises a method of treating salivary cancer in a human patient comprising subcutaneously administering about 280 mg to about 450 mg of an anti-PD-1 antibody (e.g., pembrolizumab), or antigen binding fragment thereof, to the patient once approximately every three weeks.

In a twenty-fifth embodiment (Embodiment E25), the invention comprises a method of treating cancer in a human patient comprising subcutaneously administering about 280 mg to about 450 mg of an anti-PD-1 antibody (e.g., pembrolizumab), or antigen binding fragment thereof, to the patient once approximately every three weeks, wherein the cancer is selected from the group consisting of: melanoma, non-small cell lung cancer (NSCLC), head and neck squamous cell cancer (HNSCC), relapsed or refractory classical Hodgkin lymphoma (cHL), primary mediastinal large B-cell lymphoma (PMBCL), urothelial carcinoma, microsatellite instability-high or mismatch repair deficient colorectal cancer, gastric cancer, esophageal cancer, cervical cancer, hepatocellular carcinoma, merkel cell carcinoma, renal cell carcinoma, endometrial carcinoma, TMB-H cancer, cutaneous squamous cell carcinoma, and triple-negative breast cancer.

In a sub-embodiment of Embodiment 25 (Embodiment 25B), the invention comprises a method of treating cancer in a human patient comprising subcutaneously administering about 280 mg to about 450 mg of an anti-PD-1 antibody (e.g., pembrolizumab), or antigen binding fragment thereof, to the patient once approximately every three weeks, wherein the cancer is selected from the group consisting of: melanoma, non-small cell lung cancer, relapsed or refractory classical Hodgkin lymphoma, primary mediastinal large B-cell lymphoma, head and neck squamous cell cancer, urothelial carcinoma, esophageal cancer, gastric cancer, cervical cancer, PMBCL, MSI-H cancer, hepatocellular carcinoma, Merkel cell carcinoma, renal cell carcinoma, endometrial carcinoma, TMB-H cancer, cutaneous squamous cell carcinoma, and triple-negative breast cancer.

In a twenty-sixth embodiment (Embodiment E26), the invention comprises a method of treating cancer in a human patient comprising subcutaneously administering about 280 mg to about 450 mg of an anti-PD-1 antibody (e.g., pembrolizumab), or antigen binding fragment thereof, to the patient once approximately every three weeks, wherein the cancer is a Heme malignancy.

In a sub-embodiment of Embodiment E26, the heme malignancy is selected from the group consisting of: acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (MCL-1), myelodysplastic syndrome (MDS), non-Hodgkin lymphoma (NHL), and small lymphocytic lymphoma (SLL).

In a twenty-seventh embodiment (Embodiment E27), the invention comprises a method of treating cancer in a human patient comprising subcutaneously administering about 280 mg to about 450 mg of an anti-PD-1 antibody (e.g., pembrolizumab), or antigen binding fragment thereof, to the patient once approximately every three weeks, wherein the patient has a tumor with a high mutational burden. In sub-embodiments of Embodiment E27, the tumor is a solid tumor. In some embodiments, the patient is an adult patient. In some embodiments, the patient is a pediatric patient.

In sub-embodiments of Embodiment 27, a high mutational burden is at least about mutations per megabase of genome examined, at least about 11 mutations per megabase of genome examined, at least about 12 mutations per megabase of genome examined, or at least about 13 mutations per megabase of genome examined.

In a twenty-eighth embodiment (Embodiment E28), the invention comprises a method of treating esophageal cancer in a human patient comprising subcutaneously administering about 280 mg to about 450 mg of an anti-PD-1 antibody (e.g., pembrolizumab), or antigen binding fragment thereof, to the patient once approximately every three weeks.

In sub-embodiments of Embodiment E28, the patient progressed with one previous line of standard therapy prior to receiving the anti-PD-1 antibody, or antigen binding fragment thereof. In a further embodiment, the patient progressed with one or more lines of standard therapy prior to receiving the anti-PD-1 antibody, or antigen binding fragment thereof. In another embodiment, the patient progressed with two or more lines of standard therapy prior to receiving the anti-PD-1 antibody, or antigen binding fragment thereof. In particular embodiments, the standard therapy includes one or more of: paclitaxel, docetaxel, or irinotecan.

In sub-embodiments of Embodiment E28, the patient has advanced or metastatic adenocarcinoma or squamous cell carcinoma of the esophagus.

In sub-embodiments of Embodiment E28, the patient has advanced or metastatic Siewert type I adenocarcinoma of the esophagogastric junction.

In sub-embodiments of Embodiment E28, the patient's tumor expresses PD-L1 (Combined Positive Score [CPS]≥10).

In a twenty-ninth embodiment (Embodiment E29), the invention comprises a method of treating high-risk non-muscle invasive bladder cancer (NMIBC) in a human patient comprising subcutaneously administering 280 mg to about 450 mg of an anti-PD-1 antibody (e.g., pembrolizumab), or antigen binding fragment thereof, to the patient once every approximately three weeks. In some embodiments, the patient has NMIBC with carcinoma in situ (CIS) or CIS plus papillary disease.

In a sub-embodiment of Embodiment E29, the patient was previously treated with standard therapy prior to being treated with the anti-PD-1 antibody, or antigen binding fragment thereof. In some embodiments, the prior therapy is Bacillus Calmette-Guerin (BCG) therapy. In particular embodiments, the patient did not respond to BCG therapy. In some embodiments, the patient was ineligible for radical cystectomy or chose not to undergo radical cystectomy.

In a thirtieth embodiment (Embodiment E30), the invention comprises a method of treating cutaneous squamous cell carcinoma (cSCC) in a human patient comprising subcutaneously administering 280 mg to about 450 mg of an anti-PD-1 antibody (e.g., pembrolizumab), or antigen binding fragment thereof, to the patient once every approximately three weeks. In some embodiments, the cutaneous squamous cell carcinoma is not curable by surgery or radiation.

In a thirty-first embodiment (Embodiment E31), the invention comprises a method of treating endometrial carcinoma in a human patient comprising subcutaneously administering 280 mg to about 450 mg of an anti-PD-1 antibody (e.g., pembrolizumab), or antigen binding fragment thereof, to the patient once every approximately three weeks.

In some embodiments, the method further comprises treating the patient with lenvatinib. In some embodiments, the endometrial carcinoma is advanced endometrial carcinoma that is not MSI-H or mismatch repair deficient (dMMR). In some embodiments, the patient had disease progression following prior systemic therapy.

In some sub-embodiments of Embodiment E31, the method further comprises treating the patient with lenvatinib and the endometrial carcinoma is advanced endometrial carcinoma that is not MSI-H or mismatch repair deficient (dMMR). In some embodiments, the patient had disease progression following prior systemic therapy.

In some sub-embodiments of Embodiment E31, the endometrial carcinoma is advanced endometrial carcinoma that is MSI-H or dMMR, as determined by an FDA-approved test, wherein the patient has had disease progression following prior systemic therapy in any setting. In some embodiments, the patient is not a candidate for curative surgery or radiation.

In a thirty-second embodiment (Embodiment E32), the invention comprises a method of treating cervical carcinoma in a human patient comprising subcutaneously administering 280 mg to about 450 mg of an anti-PD-1 antibody (e.g., pembrolizumab), or antigen binding fragment thereof, to the patient once every approximately three weeks.

In sub-embodiments of Embodiment E32, the method further comprises treating the patient with chemotherapy, with or without bevacizumab. In some embodiments, the cervical cancer is persistent, recurrent, or metastatic cervical cancer and the patient's tumor expresses PD-L1 (CPS≥1).

In sub-embodiments of Embodiment E32, the method further comprises treating the patient with chemotherapy, with or without bevacizumab. In some embodiments, the cervical cancer is persistent, recurrent, or metastatic cervical cancer and the patient's tumor expresses PD-L1 (CPS≥1).

In sub-embodiments of Embodiment E32, the cervical cancer is recurrent or metastatic cervical cancer with disease progression on or after chemotherapy, the patient's tumor expresses PD-L1 (CPS≥1), and the patient is not treated with chemotherapy.

In any of the methods of the invention described above (including Embodiments E1-E32), the anti-PD-1 antibody, or antigen binding fragment thereof, is any of the antibodies or antigen-binding fragments described in Section II of the Detailed Description of the Invention “PD-1 Antibodies and Antigen Binding Fragments Useful in the Invention” herein. In some embodiments, the anti-PD-1 antibody is pembrolizumab, or an antigen-binding fragment thereof, or an antibody which cross competes with pembrolizumab for binding to human PD-1. In some embodiments, the anti-PD-1 antibody is a variant of pembrolizumab.

In any of the methods of the invention described above (including Embodiments E1-E32), the anti-PD-1 antibody, or antigen binding fragment thereof, is subcutaneously administered to the patient once approximately every three weeks. In particular embodiments, the anti-PD-1 antibody, or antigen binding fragment thereof, is subcutaneously administered to the patient every three weeks, every three weeks±5 days, ±4 days, ±3 days, +2 days or ±1 day.

In any one of the methods of the invention described above (including Embodiments E1-E32), the amount of anti-PD-1 antibody or antigen-binding fragment thereof administered to the patient is from about 280 mg to about 450 mg. In further embodiments, the amount of anti-PD-1 antibody or antigen-binding fragment thereof administered to the patient is from about 280 mg to about 450 mg. In further embodiments, the amount of anti-PD-1 antibody or antigen-binding fragment is about 300 mg to about 450 mg. In further embodiments, the amount of anti-PD-1 antibody or antigen-binding fragment is about 320 mg to about 450 mg. In further embodiments, the amount of anti-PD-1 antibody or antigen-binding fragment is about 340 mg to about 450 mg. In further embodiments, the amount of anti-PD-1 antibody or antigen-binding fragment is about 360 mg to about 450 mg. In further embodiments, the amount of anti-PD-1 antibody or antigen-binding fragment is about 370 mg to about 450 mg. In further embodiments, the amount of anti-PD-1 antibody or antigen-binding fragment is about 375 mg to about 450 mg. In further embodiments, the amount of anti-PD-1 antibody or antigen-binding fragment is about 300 mg to about 430 mg. In further embodiments, the amount of anti-PD-1 antibody or antigen-binding fragment is about 320 mg to about 430 mg. In further embodiments, the amount of anti-PD-1 antibody or antigen-binding fragment is about 340 mg to about 430 mg. In further embodiments, the amount of anti-PD-1 antibody or antigen-binding fragment is about 360 mg to about 430 mg. In further embodiments, the amount of anti-PD-1 antibody or antigen-binding fragment is about 370 mg to about 430 mg. In further embodiments, the amount of anti-PD-1 antibody or antigen-binding fragment is about 375 mg to about 430 mg. In further embodiments, the amount of anti-PD-1 antibody or antigen-binding fragment is about 320 mg to about 420 mg. In further embodiments, the amount of anti-PD-1 antibody or antigen-binding fragment is about 340 mg to about 420 mg. In further embodiments, the amount of anti-PD-1 antibody or antigen-binding fragment is about 360 mg to about 420 mg. In further embodiments, the amount of anti-PD-1 antibody or antigen-binding fragment is about 370 mg to about 420 mg. In further embodiments, the amount of anti-PD-1 antibody or antigen-binding fragment is about 345 mg to about 415 mg. In further embodiments, the amount of anti-PD-1 antibody or antigen-binding fragment is about 300 mg to about 410 mg. In further embodiments, the amount of anti-PD-1 antibody or antigen-binding fragment is about 320 mg to about 410 mg. In further embodiments, the amount of anti-PD-1 antibody or antigen-binding fragment is about 340 mg to about 410 mg. In further embodiments, the amount of anti-PD-1 antibody or antigen-binding fragment is about 350 mg to about 410 mg. In further embodiments, the amount of anti-PD-1 antibody or antigen-binding fragment is about 360 mg to about 410 mg. In further embodiments, the amount of anti-PD-1 antibody or antigen-binding fragment is about 370 mg to about 410 mg. In further embodiments, the amount of anti-PD-1 antibody or antigen-binding fragment is about 375 mg to about 410 mg. In further embodiments, the amount of anti-PD-1 antibody or antigen-binding fragment is about 355 mg to about 405 mg. In further embodiments, the amount of anti-PD-1 antibody or antigen-binding fragment is about 360 mg to about 400 mg. In further embodiments, the amount of anti-PD-1 antibody or antigen-binding fragment is about 365 mg to about 395 mg. In further embodiments, the amount of anti-PD-1 antibody or antigen-binding fragment is about 300 mg to about 390 mg. In further embodiments, the amount of anti-PD-1 antibody or antigen-binding fragment is about 320 mg to about 390 mg. In further embodiments, the amount of anti-PD-1 antibody or antigen-binding fragment is about 340 mg to about 390 mg. In further embodiments, the amount of anti-PD-1 antibody or antigen-binding fragment is about 360 mg to about 390 mg. In further embodiments, the amount of anti-PD-1 antibody or antigen-binding fragment is about 370 mg to about 390 mg. In further embodiments, the amount of anti-PD-1 antibody or antigen-binding fragment is about 375 mg to about 390 mg. In further embodiments, the amount of anti-PD-1 antibody or antigen-binding fragment is about 365 mg to about 395 mg. In further embodiments, the amount of anti-PD-1 antibody or antigen-binding fragment is about 375 mg to about 385 mg. In further embodiments, the amount of anti-PD-1 antibody or antigen-binding fragment is about 379 mg to about 381 mg. In further embodiments, the amount of anti-PD-1 antibody or antigen-binding fragment is about 380 mg. In further embodiments, the amount of anti-PD-1 antibody or antigen-binding fragment is 380 mg.

In any one of the methods of the invention described above (including Embodiments E1-E32), the amount of anti-PD-1 antibody or antigen binding fragment thereof administered is 280 mg. In one embodiment, the amount of anti-PD-1 antibody or antigen binding fragment thereof administered is 285 mg. In another embodiment, the amount of anti-PD-1 antibody or antigen binding fragment thereof administered is 320 mg. In another embodiment, the amount of anti-PD-1 antibody or antigen binding fragment thereof administered is 340 mg. In another embodiment, the amount of anti-PD-1 antibody or antigen binding fragment thereof administered is 360 mg. In another embodiment, the amount of anti-PD-1 antibody or antigen binding fragment thereof administered is 370 mg. In another embodiment, the amount of anti-PD-1 antibody or antigen binding fragment thereof administered is 380 mg. In another embodiment, the amount of anti-PD-1 antibody or antigen binding fragment thereof administered is 400 mg. In another embodiment, the amount of anti-PD-1 antibody or antigen binding fragment thereof administered is 420 mg.

In any one of the methods of the invention described above (including Embodiments E1-E32), the anti-PD-1 antibody or antigen binding fragment thereof administered as a composition comprising the anti-PD-1 antibody or antigen-binding fragment thereof. For example, WO 2018/204368, the contents of which are hereby incorporated by reference, describes the preparation of liquid compositions comprising pembrolizumab.

In one embodiment, the composition comprises 130 mg/ml of the anti-PD-1 antibody or antigen binding fragment thereof. In other embodiments, the composition comprises 165 mg/ml of the anti-PD-1 antibody or antigen binding fragment thereof.

In further embodiments, the composition further comprises L-methionine. In particular embodiments, the L-methionine is present in a concentration of about 10 mM.

In further embodiments, the composition further comprises histidine buffer at about pH 5.0 to pH 6.0. In particular embodiments, the histidine is present in a concentration of about 10 mM.

In further embodiments, the composition further comprises sucrose. In particular embodiments, the sucrose is present in a concentration of about 70 mg/mL. In particular embodiments, the sucrose is present at a concentration of 7% (w/v).

In further of the invention, the composition further comprises polysorbate 80. In particular embodiments, the polysorbate 80 is present in a concentration of about 0.2 mg/mL. In particular embodiments, the polysorbate 80 is present at a concentration of 0.02% (w/v).

In some embodiments, the composition comprises 10 mM L-methionine, 10 mM histidine, pH 5.5, 7% sucrose, 0.02% polysorbate 80, and 130 mg/mL of the anti-PD-1 antibody or antigen-binding fragment thereof.

In some embodiments, the composition comprises 10 mM L-methionine, 10 mM histidine, pH 5.5, 7% sucrose, 0.02% polysorbate 80, and 165 mg/mL of the anti-PD-1 antibody or antigen-binding fragment thereof.

In some embodiments, the administration of an anti-PD-1 antibody, or antigen binding fragment thereof, can be by any suitable route, and can be facilitated by agents such as hyaluronan degrading enzymes, including hyaluronidases, including soluble PH20 polypeptides, and variants thereof. For systemic administration, the facilitating agents can be modified to increase pharmacological properties, such as serum half-life, by modifying the agents, such as with polymers. See, e.g., U.S. Pat. Nos. 7,767,429, 8,431,380, 7,871,607, International Publication No. WO 2020/022791, U.S. Patent Publication No. US2006/0104968 and European Patent 1858926, and in numerous other patents and publications. Exemplary of such agents is the known agent PEGPH20 or rHuPH20. Accordingly, specific embodiments of the methods of the invention comprise methods of treating a human patient comprising subcutaneous administration of a pharmaceutical composition comprising an anti-PD-1 antibody, or antigen binding fragment thereof, and any one of a hyaluronan degrading enzyme, hyaluronidase, soluble PH20 polypeptide, or a variant of any of the foregoing. In particular embodiments, the pharmaceutical composition comprises an anti-PD-1 antibody, or antigen binding fragment thereof, and a soluble PH20 polypeptide or a variant thereof.

In some embodiments of the methods described herein, the anti-PD-1 antibody, or antigen binding fragment thereof, is administered subcutaneously in one or more injections. In some embodiments, the anti-PD-1 antibody, or antigen binding fragment thereof, is administered in 2 injections.

In one embodiment, 380 mg of the anti-PD-1 antibody, or antigen binding fragment thereof, is administered subcutaneously as a composition comprising 130 mg/mL in one injection. In one embodiment, 380 mg of the anti-PD-1 antibody, or antigen binding fragment thereof, is administered subcutaneously in two injections.

In one embodiment, 380 mg of the anti-PD-1 antibody, or antigen binding fragment thereof, is administered subcutaneously as a composition comprising 165 mg/mL in one injection. In one embodiment, 380 mg of the anti-PD-1 antibody, or antigen binding fragment thereof, is administered subcutaneously as a composition comprising 165 mg/mL in two injections. In further embodiments, 190 mg of the anti-PD-1 antibody, or antigen binding fragment thereof, is administered subcutaneously in each of the two injections. In a further embodiment, 1.15 mL of the composition comprising 165 mg/mL of the anti-PD-1 antibody, or antigen binding fragment thereof, is administered subcutaneously in each of the two injections.

In any of the above methods described herein, the anti-PD-1 antibody, or antigen binding fragment thereof, is pembrolizumab. In any of the above methods described herein, the anti-PD-1 antibody, or antigen binding fragment thereof, is a pembrolizumab variant.

In any of the methods described herein, including Embodiment E1-E32, and sub-embodiments thereof, the method may further comprise administering one or more “additional therapeutic agents” (as used herein, “additional therapeutic agent” refers to an additional agent relative to the anti-PD-1 antibody or antigen-binding fragment thereof). The additional therapeutic agent may be, e.g., a chemotherapeutic, a biotherapeutic agent (including but not limited to antibodies to CTLA4, VEGF, EGFR, Her2/neu, VEGF receptors, other growth factor receptors, CD20, CD40, CD-40L, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods of the invention, the method further comprises administering an additional therapeutic agent. In particular embodiments, the additional therapeutic agent is an anti-CTLA4 antibody or antigen binding fragment thereof, an anti-LAG3 antibody or antigen binding fragment thereof, an anti-GITR antibody, or antigen binding fragment thereof, an anti-TIGIT antibody, or antigen binding fragment thereof, an anti-CD27 antibody or antigen binding fragment thereof, an anti-ILT3 antibody, or antigen binding fragment thereof, or an anti-ILT4 antibody, or antigen binding fragment thereof. In one embodiment, the additional therapeutic agent is a Newcastle disease viral vector expressing IL-12. In a further embodiment, the additional therapeutic agent is dinaciclib. In another embodiment, the additional therapeutic agent is navarixin. In a further embodiment, the additional therapeutic agent is vicriviroc.

In a further embodiment, the additional therapeutic agent is an oncolytic virus. In one embodiment, the additional therapeutic agent is Coxsackievirus or CVA21. In one embodiment, the additional therapeutic agent is CAVATAK™ In yet another embodiment, the additional therapeutic agent is a STING agonist.

In a further embodiment, the additional therapeutic agent is an IL-27 antagonist. In one embodiment, the additional therapeutic agent is a PARP inhibitor. In one embodiment, the additional therapeutic agent is a multi-kinase inhibitor. In one embodiment, the additional therapeutic agent is a MEK inhibitor. In one embodiment, the additional therapeutic agent is a 4-1BB agonist.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammalI and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

In some embodiments which comprise a step of administering an additional therapeutic agent (i.e., in addition to the anti-PD-1 antibody (e.g., pembrolizumab) or antigen-binding fragment thereof), the additional therapeutic agent in the combination therapy may be administered using the same dosage regimen (dose, frequency and duration of treatment) that is typically employed when the agent is used as monotherapy for treating the same cancer. In other embodiments, the patient receives a lower total amount of the additional therapeutic agent in the combination therapy than when that agent is used as monotherapy, e.g., smaller doses, less frequent doses, and/or shorter treatment duration.

The additional therapeutic agent in a combination therapy can be administered orally, intratumorally, or parenterally, including the intravenous, intramuscular, intraperitoneal, subcutaneous, rectal, topical, and transdermal routes of administration. For example, the combination treatment may comprise an anti-PD-1 antibody or antigen binding fragment thereof, and an anti-CTLA antibody or antigen binding fragment thereof, both of which may be administered intravenously or subcutaneously, as well as a chemotherapeutic agent, which may be administered orally.

A combination therapy of the invention may be used prior to or following surgery to remove a tumor and may be used prior to, during, or after radiation therapy. A combination therapy of the invention may also be used when a patient's tumor is non-resectable.

In some embodiments, a combination therapy of the invention is administered to a patient who has not been previously treated with a biotherapeutic or chemotherapeutic agent, i.e., is treatment-naive. In other embodiments, the combination therapy is administered to a patient who failed to achieve a sustained response after prior therapy with a biotherapeutic or chemotherapeutic agent, i.e., is treatment-experienced.

A combination therapy of the invention may be used to treat a tumor that is large enough to be found by palpation or by imaging techniques well known in the art, such as MRI, ultrasound, or CAT scan. In some embodiments, a combination therapy of the invention is used to treat an advanced stage tumor having dimensions of at least about 200 mm³, 300 mm³, 400 mm³, 500 mm³, 750 mm³, or up to 1000 mm³.

In some embodiments, a combination therapy of the invention is administered to a human patient who has a cancer that expresses PD-L1. In some embodiments, PD-L1 expression is detected using a diagnostic anti-human PD-L1 antibody, or antigen binding fragment thereof, in an IHC assay on an FFPE or frozen tissue section of a tumor sample removed from the patient. A patient's physician may order a diagnostic test to determine PD-L1 expression in a tumor tissue sample removed from the patient prior to initiation of treatment with the anti-PD-1 antibody, or antigen-binding fragment thereof, but it is envisioned that the physician could order the first or subsequent diagnostic tests at any time after initiation of treatment, such as for example after completion of a treatment cycle.

### IV. KITS

The invention also relates to a kit for treating a patient with cancer, the kit comprising: (a) a composition for subcutaneous injection comprising about 280 mg to about 450 mg of an anti-PD-1 antibody or antigen binding fragment thereof, and (b) instructions for using the anti-PD-1 antibody, or antigen binding fragment thereof, in any of the methods for treating cancer described herein.

The kits of the invention may provide the anti-PD-1 antibody, or antigen-binding fragment thereof, in a container and includes a package insert. The container contains at least about 280 mg to about 450 mg of a composition comprising an anti-PD-1 antibody, or antigen binding fragment thereof, and the package insert, or label, which comprises instructions for treating a patient with cancer using the composition. The container may be comprised of any shape and/or material (e.g., plastic or glass). For example, the container might be a vial, syringe or bottle. The kit may further comprise other materials that may be useful in administering the medicaments, such as needles and syringes. In some embodiments of the kit, the instructions state that the medicament is intended for use in treating a patient as described in any of Embodiments E1-E32 above in Section III entitled Methods and Uses of the Invention.

In one embodiment, the composition comprises 130 mg/ml of the anti-PD-1 antibody or antigen binding fragment thereof. In other embodiments, the composition comprises 165 mg/ml of the anti-PD-1 antibody or antigen binding fragment thereof.

In further embodiments, the composition further comprises L-methionine. In particular embodiments, the L-methionine is present in a concentration of about 10 mM.

In further embodiments, the composition further comprises histidine buffer at about pH 5.0 to pH 6.0. In particular embodiments, the histidine is present in a concentration of about 10 mM.

In further embodiments, the composition further comprises sucrose. In particular embodiments, the sucrose is present in a concentration of about 70 mg/mL. In particular embodiments, the sucrose is present at a concentration of 7% (w/v).

In further of the invention, the composition further comprises polysorbate 80. In particular embodiments, the polysorbate 80 is present in a concentration of about 0.2 mg/mL. In particular embodiments, the polysorbate 80 is present at a concentration of 0.02% (w/v).

In some embodiments, the composition comprises 10 mM L-methionine, 10 mM histidine, pH 5.5, 7% sucrose, 0.02% polysorbate 80, and 130 mg/mL of the anti-PD-1 antibody or antigen-binding fragment thereof.

In some embodiments, the composition comprises 10 mM L-methionine, 10 mM histidine, pH 5.5, 7% sucrose, 0.02% polysorbate 80, and 165 mg/mL of the anti-PD-1 antibody or antigen-binding fragment thereof.

In one embodiment, the composition is contained in a vial. In another embodiment, the composition is contained in one or more pre-filled syringes. In one embodiment, the composition is contained in two pre-filled syringes. In one embodiment, each pre-filled syringe contains 190 mg of the composition comprising the anti-PD-1 antibody or antigen binding fragment thereof. In one embodiment, each pre-filled syringe contains 1.15 mL of a composition comprising 165 mg/ml of the anti-PD-1 antibody or antigen binding fragment thereof.

In any of the kits of the invention, the anti-PD-1 antibody or antigen binding fragment can be any of the antibodies or antigen-binding fragments described in Section II of the Detailed Description of the Invention “PD-1 Antibodies and Antigen Binding Fragments Useful in the Invention”. In one embodiment, the anti-PD-1 antibody, or antigen binding fragment thereof, is pembrolizumab. In another embodiment, the anti-PD-1 antibody, or antigen binding fragment thereof, is a pembrolizumab variant.

These and other aspects of the invention, including the exemplary specific embodiments listed below, will be apparent from the teachings contained herein.

### GENERAL METHODS

Standard methods in molecular biology are described Sambrook, Fritsch and Maniatis (1982 & 1989 2^{nd} Edition, 20013^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

All publications mentioned herein are incorporated by reference for the purpose of describing and disclosing methodologies and materials that might be used in connection with the invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### Example 1

### Population PK Model Development

Pembrolizumab is currently approved for use in multiple cancer indications at a dose of either 200 mg or 2 mg/kg Q3W or 400 mg Q6W administered as an IV infusion. An alternative subcutaneous formulation would provide convenience and flexibility to patients and prescribers. A Phase I randomized clinical study designed to estimate the relative bioavailability of two different concentrations of SC formulations of pembrolizumab was performed. The relative bioavailability of two different subcutaneous formulations of pembrolizumab (concentration 165 mg/ml and 130 mg/ml) was estimated in this study using the subcutaneous dose (285 mg, given at two different concentration/volumes (as shown in Table 4) of pembrolizumab compared with the IV dose of pembrolizumab (200 mg)). Patients with advanced melanoma were randomized during the first 3 treatment cycles to receive (in a cross-over design) one dose each of: an IV infusion of 200 mg of pembrolizumab, and two SC injections of 285 mg of pembrolizumab (using one each of the two tested SC formulations, see Table 4 below). PK model-based simulations using the estimated bioavailability and between-subject variability from this study indicate that a subcutaneous dose of pembrolizumab of 380 mg Q3W should lead to comparable exposure as the approved dose of 200 mg Q3W of pembrolizumab IV.

### Study Design

Eligible patients were ≥18 years of age, had unresectable Stage III or IV melanoma not amenable to local therapy, measurable disease per RECIST v1.1, an Eastern Cooperative Oncology Group performance status of 0 or 1, and had not received prior therapy for advanced disease (except BRAF/MEK inhibitor for BRAF^{V600} mutant disease and prior adjuvant or neoadjuvant therapy received ≥4 weeks from randomization).

Patients in cohort A were randomly assigned in a crossover design to one of six treatment arms to receive a SC injection of 1.7 mL of a 165 mg/mL pembrolizumab formulation (dose 285 mg), a SC injection of 2.2 mL of a 130 mg/mL pembrolizumab formulation (dose 285 mg), and an IV infusion of pembrolizumab 200 mg over the first 3 cycles of treatment, followed by pembrolizumab 200 mg IV for ≤35 cycles of treatment. Patients in cohort A who received one of the formulations listed in Table 4 receive pembrolizumab 285 mg subcutaneous Q3W at different strengths: 130 mg/mL and 165 mg/mL.

TABLE 4
Formulations

| | |
|---|---|
| 130 mg/mL pembrolizumab | 165 mg/ml pembrolizumab |
| 7% w/v sucrose | 7% w/v sucrose |
| 0.02% w/v polysorbate 80 | 0.02% w/v polysorbate 80 |
| 10 mM histidine buffer | 10 mM histidine buffer |
| 10mM L-methionine | 10mM L-methionine |

Serum concentration data from 32 subjects collected through cycles 1 (i.e. weeks 0-3), 2 (i.e. weeks 4-6), and 3 (i.e. weeks 7-9) from a Phase I clinical trial were used to characterize the PK of SC pembrolizumab, along with extensive historical pembrolizumab IV PK data using population PK analysis. Non-linear mixed-effects modeling with Bayesian methods was applied to the Phase 1 data with priors from the previously established pembrolizumab reference PK model. The reference pembrolizumab PK model was based on pembrolizumab PK data collected from 2993 patients with various cancers who received pembrolizumab doses of 1 to 10 mg/kg Q2W, 2 to 10 mg/kg Q3W, or 200 mg Q3W in Phase I or Phase III clinical studies. The absorption phase PK parameters were estimated for SC administration from the Phase I data, and any differences between the two SC formulation strengths were also evaluated. Distribution and elimination parameters (Clearance (CL), central volume of distribution (Vc), inter-compartmental clearance (Q), and peripheral volume of distribution (Vp)) were estimated using the Phase 1 data and weakly informative priors from the reference IV model, since these phases are expected to be similar for IV and SC administrations. Given the small sample size and short duration of the SC administrations in the study i.e. two treatment cycles, parameters describing the time-dependency and effects of patient baseline characteristics on pembrolizumab PK were fixed from the previously established reference IV PK model.

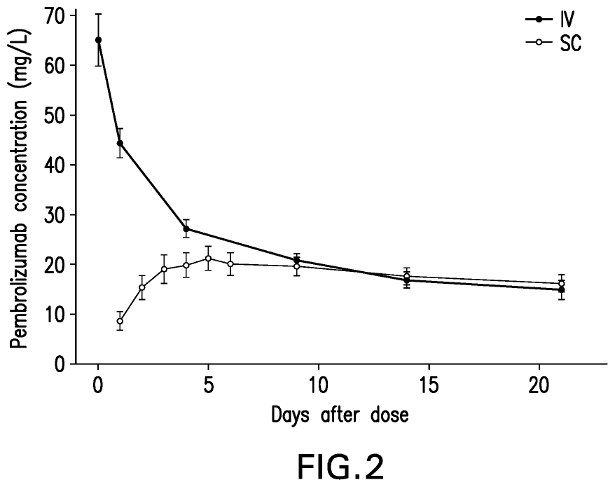

The new population PK model was able to simultaneously describe pembrolizumab PK after IV or SC administrations. The final parameter estimates of the combined SC and IV population PK model are displayed in Table 5. The absorption phase for SC administration was characterized by a first order absorption rate (ka) and bioavailability (F) parameters. Distribution and elimination phases were described by a two-compartment model with time-dependent clearance and a fixed effect of body weight as established historically in the reference pembrolizumab PK model. Inclusion of a covariate effect for the strength of SC formulation or use of distinct absorption models for each SC formulation were not statistically significant, indicating no meaningful difference in the bioavailability and absorption rate between the two SC formulations. Goodness of fit evaluation demonstrated the absence of structural bias as a function of drug concentration or time. Thus, the analysis showed that the two SC formulation strengths of pembrolizumab behaved similar in their PK, with an estimated bioavailability of 66% (95% CI. 58% to 74%). Mean time to achieve maximum serum concentration with pembrolizumab SC was estimated to be 5.5 days (range, 3 to 14 days; FIG. 2). In addition, no anti-drug antibodies (ADA) were observed in the Phase I study.

TABLE 5

| | | | |
|---|---|---|---|
| Parameter | Value | % RSE | % CV |

Estimated parameters using KEYNOTE -555 data

| | | | |
|---|---|---|---|
| Ka (/day) | 0.191 | 13.1 | 72.2 |
| F (bioavailability) | 0.66 | 6.08 | 28.2 |
| CL (L/day) | 0.25 | 6.28 | 27.5 |
| Vc (L) | 3.39 | 5.68 | 24.3 |
| Q (L/day) | 0.631 | 14.2 | 27.5 |
| Vp (L) | 2.42 | 1.03 | 24.3 |
| Residual error | 0.161 | 2.89 | |

Fixed parameters from reference IV model

| | | | |
|---|---|---|---|
| Maximum effect of time on CL | −0.218 | — | 79.5 |
| TI₅₀ (day) | 65.5 | — | |
| Hill | 2.99 | — | |
| α for CL and Q (allometric scaling factor) | 0.534 | — | |
| α for Vc and Vp (allometric scaling factor) | 0.514 | — | |
| Albumin effect on CL | −0.849 | — | |
| eGFR effect on CL | 0.123 | — | |
| Sex effect on CL | −0.162 | — | |
| Baseline ECOG effect on CL | −0.0697 | — | |
| Baseline tumor size effect on CL | 0.0933 | — | |
| Bilirubin effect on CL | −0.0488 | — | |
| Albumin effect on Vc | −0.233 | — | |
| Sex effect on Vc | −0.131 | — | |
| Tumor type effect (NSCLC vs other) on Vc | −0.059 | — | |

CV, coefficient of variation of between-subject distributions of parameters; eGFR, estimated glomerular filtration rate; NSCLC, non-small cell lung cancer; RSE, relative standard error; TI₅₀, time at which 50% of the maximum effect on clearance has been achieved.
Fixed parameters from reference IV model
^{a}Presented population mean parameter estimates represent a typical patient with mean baseline characteristics.

The results of the Phase 1 study showed the SC pembrolizumab was well tolerated over the first 3 cycles of treatment; skin and subcutaneous disorders were mostly mild to moderate (data not shown). Erythema was reported by 3 patients when they received pembrolizumab 130 mg/mL SC and 2 patients when they received pembrolizumab 165 mg/mL SC.

The two tested SC formulation strengths of pembrolizumab (130 mg/ml and 165 mg/ml) had similar absorption PK. SC administration of pembrolizumab was well tolerated with no ADAs or significant injection-site reactions. An estimated bioavailability for subcutaneous pembrolizumab is estimated at 660% (9500 CI, 5800 to 740%).

### Example 2

### A Three-Weekly (Q3W) Dosing Schedule for Pembrolizumab Across Multiple Tumor Types Based on an Evaluation Using Modeling and Simulation

Pembrolizumab, an anti-PD-1 checkpoint inhibitor currently approved for use in multiple cancer indications, has demonstrated safety and efficacy when administered at a dose of either 200 mg or 2 mg/kg Q3W. The robust characterization of pembrolizumab pharmacokinetics (PK) and exposure (concentration)-response (E-R) relationships for both efficacy and safety allow the use of model-based approaches to support alternative routes of administration for pembrolizumab.

The selected dosing regimen of SC pembrolizumab is 380 mg Q3W. PK model-based simulations indicate that the 380 mg Q3W dose should lead to comparable exposure as the approved dose of 200 mg Q3W of pembrolizumab administered via IV. In principle, similar PK exposures lead to similar efficacy and safety of pembrolizumab, given that the exposure-response relationships for both efficacy and safety are already well established for pembrolizumab.

Typically, bioequivalence is ascertained between a new proposed formulation/route of administration compared to a previously approved formulation/route of the same drug, by establishing that the PK exposure is no more than 20% deviant from the reference/approved formulation/route, which is not expected to have any clinically meaningful impact on efficacy or safety. For the lower end of exposures, establishing non-inferiority is adequate, using a generally accepted margin of a lower bound of 90% CI around GMR>0.8. This would ascertain that the efficacy of a dose of pembrolizumab administered subcutaneously (“SC pembrolizumab”) is no worse than the efficacy of the dose of pembrolizumab (200 mg Q3W) administered by an IV route of administration (“IV pembrolizumab”).

In the case of comparing SC pembrolizumab to IV pembrolizumab, it is important to note the key differences that inherently exist in the PK profiles between SC and IV administrations. Typically, for comparable doses (adjusted for bioavailability) the concentrations with SC administration gradually accrue over ˜6 days, and the peak concentrations (Cₘₐₓ) after a SC administration of pembrolizumab are much lower than the Cₘₐₓ achieved at the end of an IV infusion. Specifically, with the 380 mg Q3W SC dose, the Cₘₐₓ is expected to be much reduced (˜60% lower at Cycle 1 and ˜34% lower at steady state) than the Cₘₐₓ achieved at 200 mg Q3W IV. Thus, with the selected SC dose, there is no increase expected in Cₘₐₓ throughout treatment relative to the approved dose of 200 mg Q3W. Further, all PK exposures of 380 mg Q3W SC pembrolizumab will certainly remain well below the 5-fold higher dose/exposures at 10 mg/kg Q2W IV, which is the highest clinically evaluated dose with established safety. Therefore, the safety profile after SC administration of 380 mg Q3W SC pembrolizumab is unlikely to differ from the safety profile previously established for the 200 mg Q3W IV pembrolizumab dose identified, and hence quantitative assessment for the upper bound of exposures was not further evaluated.

PK model-based simulations were performed to select the SC dose, targeting consistency of the SC PK exposure profile with that of the approved 200 mg Q3W dose and overall exposure profiles based on clinical experience with pembrolizumab IV. The simulations were performed on the reference pembrolizumab PK dataset including 2993 subjects with melanoma or non-small cell lung cancer (NSCLC) from the pooled dataset of Phase I and Phase III trials.

Pembrolizumab serum concentrations were simulated for doses ranging from 260 mg to 420 mg Q3W of pembrolizumab SC and 200 mg Q3W of pembrolizumab IV from cycle 1 through cycle 6 (18 weeks, achieving steady state) using the combined SC and IV PK model (described in Table 5), including estimates of population mean PK parameters as well as uncertainty on these estimates. Between-subject-variability was not accounted for in these initial simulations. For each subject in the dataset, the simulated trough concentration at the end of the dosing interval (C_{trough}) and area under curve (AUC) exposure were determined both over Cycle 1 (first dose) and Cycle 6 (representing steady state). The PK parameters C_{trough} and AUC_{0-3 wks} indicate PK exposure and are regarded as drivers of pembrolizumab efficacy. Cycle 1 represents the PK exposures achieved after the first dose is administered. Cycle 6 represents the PK exposures achieved at steady state, which are the exposures that will then be maintained throughout treatment duration. The geometric mean (GM) of C_{trough} and AUC_{0-3 wks} was calculated for each SC dose and the 200 mg IV dose of pembrolizumab. Then, the geometric mean ratio (GMR) for both of these PK parameters of SC versus IV pembrolizumab (as the ratio of GM of each formulation group) and the 90% confidence interval (CI) of the GMR were calculated, for treatment cycles 1 and 6.

As a next step, to ensure robust SC dose selection, stochastic simulations were performed, including between-subject and residual variability. 100 replicate simulations were performed on the reference PK dataset (N=2993 subjects) for pembrolizumab. The PK parameters C_{trough}, AUC_{0-3 wks} and Cₘₐₓ were determined for each subject in each replicate simulated dataset, both over Cycle 1 (first dose) and at steady state. For each replicate, the GM of these PK parameters was determined at each SC dose and the 200 mg IV dose. The GMR for the simulated C_{trough} of SC versus IV was calculated at cycles 1 and 6. The median GMR from 100 replicates was determined, for each of the simulated SC doses. Similarly, the difference in GM of simulated AUC_{0-3 wks} and Cₘₐₓ (as a percentage relative to IV) was also determined for all simulated SC doses at Cycles 1 and 6.

Finally, an additional set of simulations was performed including assessment of GMR of SC:IV PK parameters through treatment duration in a clinical trial setting, to confirm the adequacy of the selected SC dose. The goal of these clinical trial simulations was to assess non-inferiority of C_{trough} of the selected SC dose versus IV pembrolizumab. The simulation scenario included 2000 replicate trials with a sample size of 228 subjects per trial (randomly sampled with replacement from the reference PK dataset of 2993 subjects) with 2:1 randomization for SC:IV (i.e., the sample size and randomization ratio chosen in the formal power calculation for non-inferiority of C_{trough} in a Phase III study). For each simulated trial, the PK parameter C_{trough} was determined for all subjects, for every treatment cycle from Cycle 1 (first dose) to cycle 6 (steady state), and then the GMR of the simulated C_{trough} of SC versus IV and the associated 90% CI were calculated, as described above. The SC:IV GMR and 90% CI bounds for each cycle were then summarized using the median values across the 2000 simulated trials.

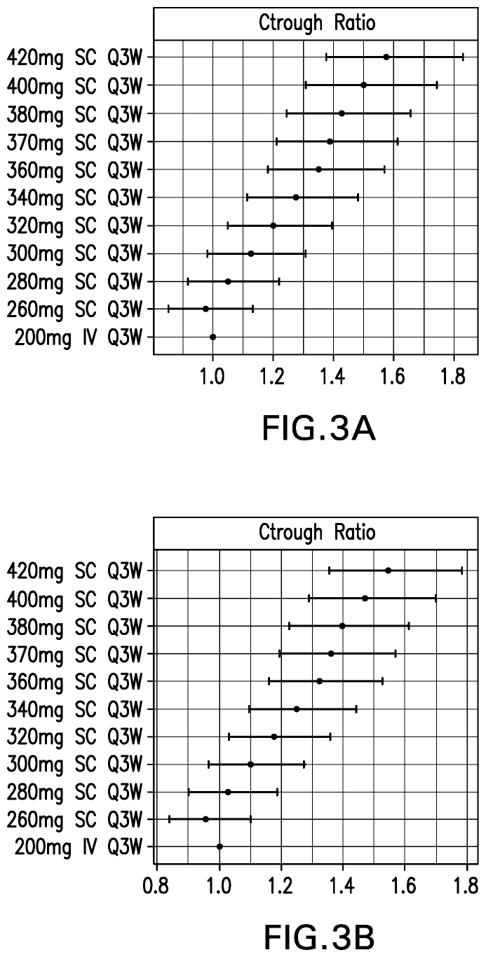

The simulations showed that doses of 280 mg through 420 mg of pembrolizumab all had a population mean SC:IV C_{trough} ratio greater than 1. See FIGS. 3A and 3B. FIG. 3A summarizes the population mean level C_{trough} across simulated SC doses at Cycle 1. FIG. 3B summarizes the population mean level C_{trough} at steady state.

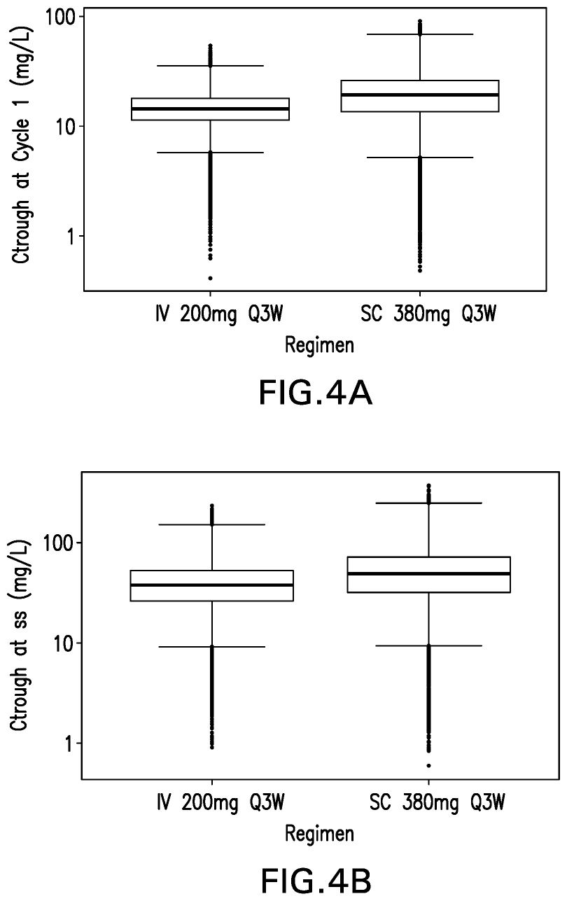

Efficacy is expected to be retained with SC pembrolizumab at the dose of 380 mg Q3W based on the following:
C_{trough} at a 380 mg Q3W SC dose is expected to be ˜30% higher than 200 mg Q3W IV, throughout treatment duration. Moreover, the distributions of C_{trough} largely overlap between SC and IV at both Cycle 1 and steady state. See FIGS. 4A and 4B.
The 380 mg Q3W SC dose led to overall comparable AUC_{0-3 wks} exposure for SC and IV administrations at Cycle 1 and steady state.

Safety is expected to be maintained with SC pembrolizumab at the dose of 380 mg based on the following:
Cₘₐₓ is expected to be much reduced (˜₆₀% lower at Cycle 1 and ˜34% lower at steady state) than the Cₘₐₓ achieved at 200 mg Q3W IV. Thus, there is no increase expected in Cₘₐₓ throughout treatment relative to the approved dose of 200 mg Q3W IV.
All SC exposures (Cₘₐₓ, C_{avg}, C_{trough}) over the dosing interval of 3 weeks and throughout duration of treatment will generally remain below the Cₘₐₓ and initial concentrations of 200 mg Q3W IV and well below the highest dose/exposures with clinical experience and established safety (i.e., 10 mg/kg Q2W).

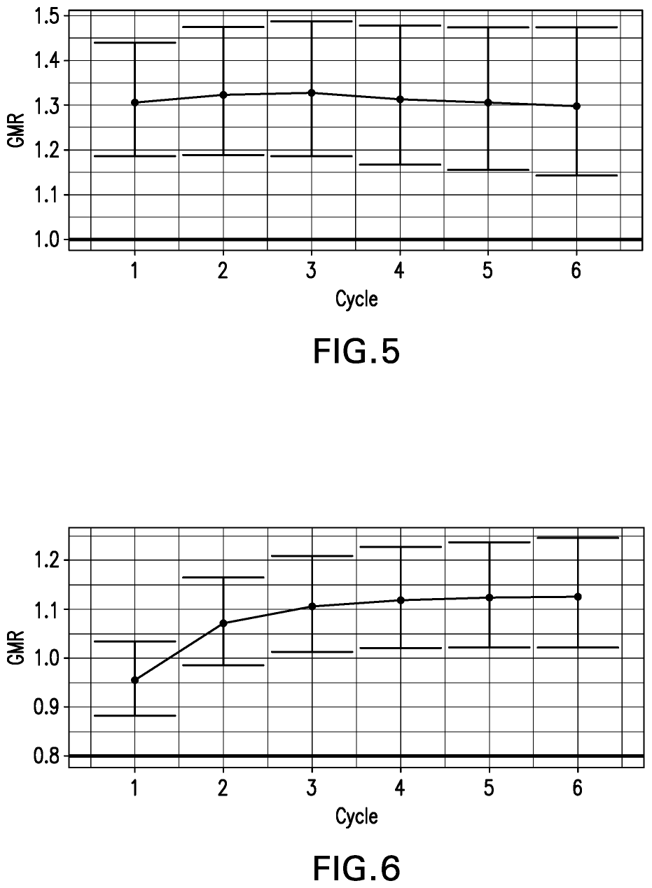

FIGS. 4A and 4B summarize the results of the population simulation including variability for C_{trough} for a dose of 380 mg Q3W SC and 200 mg IV of pembrolizumab. The simulations showed that the 380 mg SC dose leads to a range of C_{trough} across different patients that generally overlaps with the 200 mg IV dose. FIGS. 4A and 4B depict the distribution (median, 5^{th}, 25^{th}, 75^{th}, and 95^{th} percentiles) of C_{trough} at cycle 1 and steady state respectively, using PK model-based simulations at a dose of 380 mg SC and 200 mg IV of pembrolizumab. Simulated C_{trough} in 2993 subjects from 100 replicate simulated datasets are shown. FIG. 5 summarizes the results of the clinical trial simulation for C_{trough} for a dose of 380 mg Q3W SC and 200 mg IV of pembrolizumab. The simulations showed that at a dose of 380 mg Q3W of SC pembrolizumab, the GMR and lower bound of the 90% CI of GMR of SC/IV trough concentrations are >1 across all cycles from 1 to 6. FIG. 5 depicts GMR and 90% CI of SC/IV C_{trough} from cycles 1 to 6 (steady-state) using PK model-based simulations at a dose of 380 mg SC and 200 mg IV of pembrolizumab. GMR, lower and upper bound of 90% CI was determined for each trial and the 50^{th} percentile of these metrics across 2000 replicate simulated trials are shown. FIG. 6 summarizes the results of the clinical trial simulation for AUC_{0-3 wks} for a dose of 380 mg Q3W SC and 200 mg IV of pembrolizumab. The simulations showed that the 380 mg Q3W SC dose led to the GMR of SC/IV AUC exposure >0.95 and lower bound of the 90% CI of GMR of SC/IV AUC exposure >0.8 across all cycles from 1 to 6. FIG. 6 depicts GMR and 90% CI of SC/IV AUC_{0-3 wks} from cycles 1 to 6 (steady-state) using PK model-based simulations at a dose of 380 mg SC and 200 mg IV of pembrolizumab. GMR, lower and upper bound of 90% CI determined for each trial and 50^{th} percentile of these metrics across 2000 replicate simulated trials are shown.

Overall, the model-based simulations as supported by FIGS. 3A, 3B, 4A, 4B, 5 and 6 indicate that a dose of 380 mg Q3W of pembrolizumab administered SC should lead to an optimal PK exposure profile that is consistent with that of the approved dose of 200 mg Q3W of pembrolizumab IV, thus maintaining efficacy, while also remaining within the clinical safety margin

### Example 3

A Randomized, Phase 3, Open-Label Study to Investigate the Pharmacokinetics and Safety of Subcutaneous Pembrolizumab Versus Intravenous Pembrolizumab, Administered with Platinum Doublet Chemotherapy, in the First-Line Treatment of Participants with Metastatic Squamous or Nonsquamous Non-Small-Cell Lung Cancer

This clinical study will evaluate pembrolizumab SC as first-line therapy in the treatment of metastatic squamous and nonsquamous NSCLC by assessing the PK, safety, and efficacy of pembrolizumab when administered as an SC injection Q3W in combination with standard-of-care chemotherapy.

Pembrolizumab is currently indicated for the treatment of a number of tumor types including both squamous and nonsquamous NSCLC as monotherapy and in combination with chemotherapy. Adult patients are currently to be treated with pembrolizumab at an IV dose of 200 mg every 3 weeks (Q3W) or 400 mg every 6 weeks (Q6W). However, there is high demand for SC formulations, with over 80% of patients preferring SC to IV administration. A SC formulation of pembrolizumab has been developed for use as an alternative to the IV formulation of pembrolizumab. In this study, dosing of a pembrolizumab SC formulation will be achieved using a high concentration of pembrolizumab (165 mg/mL) in 2 standard prefilled syringes for a total dose of 380 mg, with each syringe containing 1.15 mL volume with 190 mg pembrolizumab. Benefits of SC administration include time savings for patients and providers, convenience, reduced administration costs, ease of administration, and reduced health care resource burden. Additionally, a SC dosing option will reduce patient chair time, thus making it feasible for infusion centers to treat more patients.

### Study Design

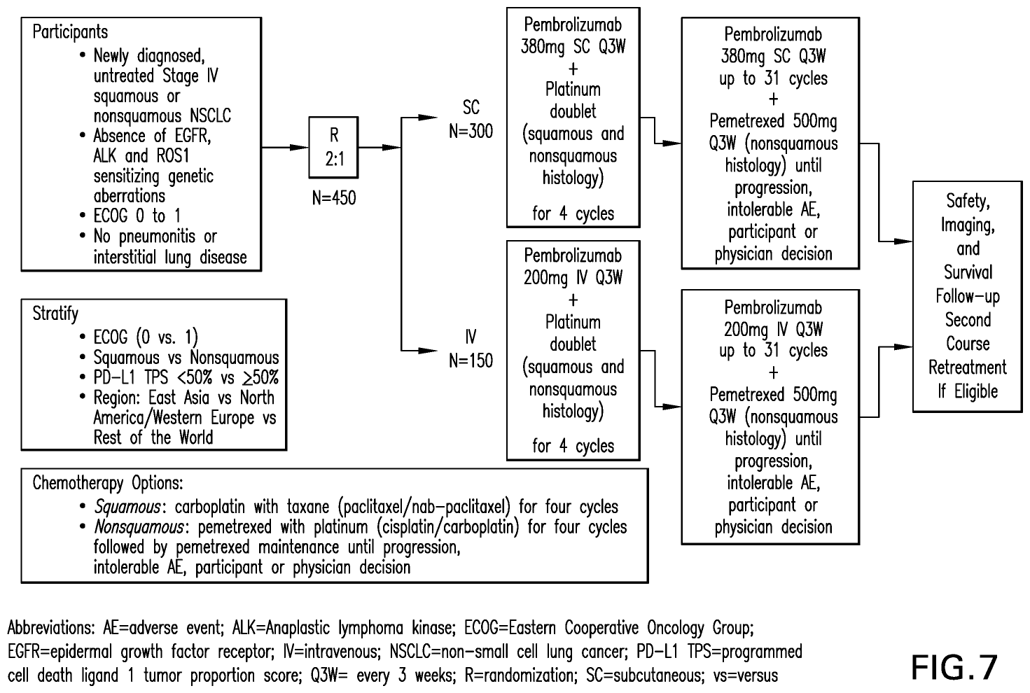

This is a Phase 3, randomized, active-controlled, parallel-group, multisite, open-label study of pembrolizumab SC administered with platinum doublet chemotherapy (Arm A) versus pembrolizumab IV administered with platinum doublet chemotherapy (Arm B) in 450 participants with metastatic squamous or nonsquamous NSCLC. Participants must have newly diagnosed, untreated Stage IV NSCLC, an ECOG PS of 0 to 1, and no current pneumonitis or interstitial lung disease at enrollment. After signing the informed consent, candidate participants will be screened against all of the eligibility criteria. Eligible participants will be randomly assigned to study intervention arm in a 2:1 ratio (Arm A to Arm B). A schematic of the study design can be found in FIG. 7. The arms of the study are as follows:
Arm A—Participants will receive up to 35 cycles of 380 mg pembrolizumab SC Q3W administered with platinum doublet chemotherapy
Arm B—Participants will receive up to 35 cycles of 200 mg pembrolizumab IV Q3W administered with platinum doublet chemotherapy
Platinum Doublet Chemotherapy:
   Participants with squamous NSCLC will receive 4 cycles of carboplatin in combination with a taxane (paclitaxel/nab-paclitaxel).
   Participants with nonsquamous NSCLC will receive 4 cycles of pemetrexed with a platinum (cisplatin/carboplatin) followed by pemetrexed maintenance until progression, an intolerable AE, or discontinuation by participant or physician decision.
      The intervention groups and duration are as follows:

| | | Dose | | Dose | Route of | Treatment |
|---|---|---|---|---|---|---|
| Group | Product | Strength | | Frequency | Administration | Period |
| Arm A | Pembrolizumab | 380 | mg | Q3W | SC | Up to 35 |
| | SC | | | | | cycles |
| Arm B | Pembrolizumab | 200 | mg | Q3W | IV | Up to 35 |
| | IV | | | | | cycles |
| Arms A & B | Paclitaxel | 200 | mg/m² | Q3W | IV | 4 cycles |
| (squamous | | | | | | |
| histology) | | | | | | |
| Arms A & B | Nab-paclitaxel | 100 | mg/m² | Days 1, 8 and | IV | 4 cycles |
| (squamous | | | | 15 of each 3- | | |
| histology) | | | | week cycle | | |
| Arms A & B | Carboplatin | Squamous: | | Q3W | IV | 4 cycles |
| (squamous | | AUC 6 | | | | |
| and non- | | mg/ml/min | | | | |
| squamous | | Non | | | | |
| histology) | | Squamous: | | | | |
| | | QUC 5 | | | | |
| | | mg/ml/min | | | | |
| Arms A & B | Cisplatin | 75 | mg/m² | Q3W | IV | 4 cycles |
| (non- | | | | | | |
| squamous | | | | | | |
| histology) | | | | | | |
| Arms A & B | Pemetrexed | 500 | mg/m² | Q3W | IV | Until |
| (non- | | | | | | progression, |
| squamous | | | | | | intolerable |
| histology) | | | | | | AE, |
| | | | | | | participant |
| | | | | | | or physician |
| | | | | | | decision. |

### Inclusion Criteria

Participants are eligible to be included in the study only if all the following criteria apply:
Participant has pathologically (histologically or cytologically) confirmed diagnosis of squamous or nonsquamous NSCLC.
Participant has Stage IV (T any, N any, M1a, M1b, or M1c—American Joint Committee on Cancer 8th Edition) squamous or nonsquamous NSCLC.
Participant has confirmation that EGFR, ALK, or ROS1-directed therapy is not indicated (documentation of absence of tumor-activating EGFR mutations AND absence of ALK and ROS1 gene rearrangements, OR presence of a KRAS mutation) in nonsquamous NSCLC as well as mixed nonsquamous/squamous NSCLC. Participants with purely squamous NSCLC do not require testing.
Participant has not received prior systemic treatment for their metastatic NSCLC.
Participants who received adjuvant or neoadjuvant therapy are eligible if the adjuvant/neoadjuvant therapy was completed at least 12 months prior to the development of metastatic disease.
Participant is at least 18 years of age
Participant has an Eastern Cooperative Oncology Group performance status (ECOG PS) of 0 or 1 (as assessed within 7 days prior to randomization)
The participant provides document informed consent for the Study
The participant has measurable disease per RECIST 1.1 as assessed by the local site investigator/radiology. Lesions situated in a previously irradiated area are considered measurable if progression has been shown in such lesions.
The participant submits an archival tumor tissue sample or newly obtained core or incisional biopsy of a tumor lesion not previously irradiated for PD-L1 status determination prior to randomization.
The participant has adequate organ function
A female participant is eligible to participate if she is not pregnant, no breastfeeding and agrees to follow specific contraceptive guidance during the treatment period and for a specified number of days thereafter.

### Study Objectives

The primary objectives of the study are to compare Cycle 1 AUC_{0-3 wks} and C_{trough} at the end of Cycle 6 for pembrolizumab SC versus pembrolizumab IV, administered with platinum doublet chemotherapy. Non-inferiority will be evaluated with the non-inferiority margin of 0.8. Efficacy will be evaluated by ORR, DOR, and PFS per RECIST 1.1 as determined by blinded independent central review (BICR), and OS.

| | Endpoint Measured |
|---|---|
| Primary Objective | |
| To compare AUC between pembrolizumab | Cycle 1 AUC_{0-3 weeks} |
| SC vs. pembrolizumab IV | |
| To compare C_{trough} between pembrolizumab | C_{trough} at the end of Cycle 6 |
| SC vs. pembrolizumab IV | |
| Secondary Objectives | |
| To evaluate pembrolizumab SC and | OR: CR or PR |
| pembrolizumab IV with respect to ORR per | |
| RECIST 1.1 as assessed by BICR | |
| To evaluate exposure of pembrolizumab SC | Cycle 1: C_{trough}, Cₘₐₓ |
| compared to pembrolizumab IV. | Cycle 6: AUC_{0-3 wks}, Cₘₐₓ |
| To evaluate the safety and tolerability of | AE |
| pembrolizumab SC compared to | Study intervention discontinuation due to AEs |
| pembrolizumab IV. | |
| To evaluate pembrolizumab SC and | PFS: the time from randomization to the first |
| pembrolizumab IV with respect to PFS per | documented disease progression or death due |
| RECIST 1.1 as assessed by BICR. | to any cause, whichever occurs first |
| To evaluate pembrolizumab SC and | OS: the time from randomization to death due |
| pembrolizumab IV with respect to OS. | to any cause |
| To evaluate pembrolizumab SC and | DOR: the time from first response (CR or PR) |
| pembrolizumab IV with respect to DOR per | to subsequent disease progression or death |
| RECIST 1.1 as assessed by BICR. | from any cause, whichever occurs first |
| To evaluate the development of ADAs | Anti-pembrolizumab antibody levels |
| following administration of pembrolizumab | |
| SC compared to pembrolizumab IV. | |

### Pharmacokinetic Parameters to Measure Non-Inferiority

AUC (or C_{avg}) exposure is a relevant PK parameter to compare between SC and IV formulations from an overall exposure and safety perspective. The Cycle 1 AUC_{0-3 wks} would be the most conservative measure of exposure to ensure non-inferiority of SC exposure relative to IV, right from the start of treatment. Hence Cycle 1 AUC_{0-3 wks} is proposed as one of the primary endpoints in this study. Cycle 1 exposures are predictive of steady state exposures; the pembrolizumab PK model may be applied to predict steady state exposures for SC based on data at the end of Cycle 1. In addition, the accumulation after multiple dosing with SC is expected to be higher than that for IV. Thus, demonstrating non-inferiority of AUC exposure at Cycle 1 would imply non-inferiority at steady state as well. Higher AUC exposure at steady state due to accumulation after the 380 mg SC dose is not expected to exceed the established clinical safety margin for pembrolizumab (i.e., exposure at 10 mg/kg Q2W). Hence, AUC exposure matching at Cycle 1 is adequate.

The pharmacological activity of mAbs is mediated through direct interaction with a specific target, and thus, target saturation can be used as a surrogate for maximal pharmacology and therapeutic activity. Pembrolizumab exerts its action through blockade of PD-1 receptors expressed on immune cells, and an efficacious dose is expected to be dictated by the level of exposure necessary to saturate the PD-1 target on immune cells. Hence, it is reasonable to expect that the therapeutic activity will be maintained as long as drug concentrations remain above that required for target saturation, regardless of formulation or dosing regimen. Exposures at the approved dose of 200 mg IV maintain target saturation, and thereby efficacy, throughout the dosing interval of 3 weeks. Therefore, C_{trough}, the concentration at the end of the dosing interval at the approved dose of 200 mg Q3W IV, may be considered a threshold, maintaining concentrations above which should maintain the efficacy of pembrolizumab. PK steady state for pembrolizumab is achieved by Cycle 6 (˜18 weeks) and exposures at Cycle 6 will be the exposures maintained through the remainder of treatment. Hence, demonstration of non-inferiority of C_{trough} at Cycle 6 would enable the inference that with SC pembrolizumab efficacy will be retained similar to that with IV.

### SC Dose

The planned dose of pembrolizumab SC for this study is 380 mg Q3W. Based on data from KEYNOTE-555 Cohort A, the bioavailability of pembrolizumab SC is estimated at 64% (95% CI. 54% to 74%). PK model-based simulations indicate that the 380 mg dose will lead to comparable exposures as the approved dose of 200 mg pembrolizumab IV (see Examples 1 & 2 above).

To ensure robust SC dose selection, both mean level and stochastic simulations (considering variability) were performed using PK parameter estimates from the combined SC and IV PK model. Pembrolizumab PK was simulated for SC doses ranging from 320 mg to 420 mg Q3W and 200 mg Q3W IV from Cycle 1 (first dose) through Cycle 6 (representing steady state), using estimates of typical values of PK parameters, covariate effects, between-participant variability and residual error. The PK parameters C_{trough}, AUC_{0-3 wks} and Cₘₐₓ were determined for each participant, both over Cycle 1 (first dose) and at steady state (Cycle 6). The GM of these PK parameters was determined at each SC dose and the 200 mg IV dose. The GMR for the simulated C_{trough} and AUC of SC versus IV was calculated at Cycles 1 and 6, for each of the simulated SC doses.

Efficacy is expected to be retained with SC pembrolizumab at the selected dose of 380 mg Q3W. Specifically, C_{trough} at a 380 mg Q3W SC dose is expected to be ˜30% higher than 200 mg Q3W IV, throughout treatment duration. Moreover, the distributions of C_{trough} largely overlap between SC and IV at both Cycle 1 and steady state. The 380 mg Q3W SC dose led to overall comparable AUC_{0-3 wks} exposure for SC and IV administrations at Cycle 1 and steady state.

Safety is expected to be maintained with SC pembrolizumab at the selected dose. Key differences inherently exist in the PK profiles between SC and IV administrations. Concentrations gradually accrue over ˜6 days after a SC administration of pembrolizumab, and the Cₘₐₓ is much lower than the Cₘₐₓ achieved at the end of an IV infusion (at bioavailability-adjusted SC doses). Specifically, with the 380 mg Q3W SC dose, the Cₘₐₓ is expected to be much reduced (˜60% lower at Cycle 1 and ˜34% lower at steady state) than the Cₘₐₓ achieved at 200 mg Q3W IV. Thus, there is no increase expected in Cₘₐₓ throughout treatment relative to the approved dose of 200 mg Q3W IV. All SC exposures (Cₘₐₓ, C_{avg}, C_{trough}) over the dosing interval of 3 weeks and throughout duration of treatment will generally remain below the Cₘₐₓ and initial concentrations of 200 mg Q3W IV and well below the highest dose/exposures with clinical experience and established safety (i.e., 10 mg/kg Q2W).

Overall, the model-based simulations indicate that a dose of 380 mg Q3W of pembrolizumab administered SC will lead to an optimal PK exposure profile that is consistent with that of the approved dose of 200 mg Q3W of pembrolizumab IV, thus maintaining efficacy, while also remaining within the clinical safety margin.

### Chemotherapy Dose

The chemotherapy treatments used in this study are well-established regimens for squamous (carboplatin with paclitaxel or nab-paclitaxel) or nonsquamous (pemetrexed and carboplatin or cisplatin) NSCLC, as described above.

What is claimed is:

1. A method of treating cancer in a human patient comprising subcutaneously administering to the patient about 280 mg to about 450 mg of an anti-programmed death 1 (anti-PD-1) antibody, or antigen binding fragment thereof, every approximately three weeks, wherein the anti-PD-1 antibody or antigen binding fragment thereof comprises:
(a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively; or
(b) light chain CDRs LC-CDR1, LC-CDR2 and LC-CDR3 comprising a sequence of amino acids as set forth in SEQ ID NOs: 11, 12 and 13, respectively and heavy chain CDRs HC-CDR1, HC-CDR2 and HC-CDR3 comprising a sequence of amino acids as set forth in SEQ ID NOs: 14, 15 and 16, respectively.

2. The method of claim 1, wherein the dose is at least 1.6 times higher than a 200 mg or a 2 mg/kg dose of the anti-PD-1 antibody, or antigen binding fragment thereof.

3. The method of claim 1, wherein the bioavailability of the anti-PD-1 antibody, or antigen binding fragment thereof, is at least 64%.

4. The method of claim 1, wherein the bioavailability of the anti-PD-1 antibody, or antigen binding fragment thereof, is at least 66%.

5. The method of claim 2, wherein the dose is 1.9 times higher than a 200 mg or a 2 mg/kg dose of the anti-PD-1 antibody, or antigen binding fragment thereof.

6. The method of claim 1, wherein the subcutaneous administration of the anti-PD-1 antibody, or antigen binding fragment thereof results in a C_{trough} that is the same as, or greater than, the C_{trough} of a 200 mg dose or a 2 mg/kg dose of the anti-PD-1 antibody, or antigen binding fragment thereof, administered by an intravenous (IV) route of administration.

7. The method of claim 6, wherein the subcutaneous administration of the anti-PD-1 antibody, or antigen binding fragment thereof, results in a ratio of subcutaneous C_{trough} to IV C_{trough} of at least 1.

8. The method of claim 7, wherein the subcutaneous administration of the anti-PD-1 antibody, or antigen binding fragment thereof, results in a ratio of subcutaneous C_{trough} to IV C_{trough} from 1.0 to 1.6.

9. The method of claim 8, wherein the dose administered by an IV route of administration is 200 mg.

10. The method of claim 1, wherein the anti-PD-1 antibody or antigen binding fragment thereof, comprises:
(a) a heavy chain variable region comprising a sequence of amino acids as set forth in SEQ ID NO: 9, or a variant of SEQ ID NO: 9, and
(b) a light chain variable region comprising:
(i) a sequence of amino acids as set forth in SEQ ID NO:4, or a variant of SEQ ID NO:4,
(ii) a sequence of amino acids as set forth in SEQ ID NO:22, or a variant of SEQ ID NO:22, or
(iii) a sequence of amino acids as set forth in SEQ ID NO:23, or a variant of SEQ ID NO:23.

11. The method of claim 1, wherein the anti-PD-1 antibody, or antigen binding fragment thereof, comprises a heavy chain variable region comprising a sequence of amino acids as set forth in SEQ ID NO:9 and a light chain variable region comprising a sequence of amino acids as set forth in SEQ ID NO:4.

12. The method of claim 1, wherein the anti-PD-1 antibody, or antigen binding fragment thereof, is a monoclonal antibody comprising:
(a) a heavy chain comprising a sequence of amino acids as set forth in SEQ ID NO: 10, or a variant of SEQ ID NO: 10, and
(b) a light chain comprising a sequence of amino acids as set forth in SEQ ID NO:5, a variant of SEQ ID NO:5, SEQ ID NO:24, a variant of SEQ ID NO:24, SEQ ID NO:25, or a variant of SEQ ID NO:25.

13. The method of claim 1, wherein the anti-PD-1 antibody, or antigen binding fragment thereof, is a monoclonal antibody comprising a heavy chain comprising a sequence of amino acids as set forth in SEQ ID NO: 10 and a light chain comprising a sequence of amino acids as set forth in SEQ ID NO: 5.

14. The method of claim 1, wherein the cancer is selected from the group consisting of: melanoma, non-small cell lung cancer, small cell lung cancer, head and neck cancer, urothelial cancer, breast cancer, gastric cancer, gastroesophageal junction adenocarcinoma, multiple myeloma, hepatocellular cancer, non-Hodgkin lymphoma, primary mediastinal large B-cell lymphoma (PMBCL), renal cancer, classical Hodgkin lymphoma, mesothelioma, ovarian cancer, esophageal cancer, anal cancer, biliary tract cancer, colorectal cancer, cervical cancer, endometrial cancer, cutaneous squamous cell cancer, thyroid cancer, prostate cancer, glioblastoma, Merkel cell carcinoma, salivary cancer, and a cancer characterized by a tumor having a high mutational burden.

15-43. (canceled)

44. The method of claim 1, wherein the anti-PD-1 antibody, or antigen binding fragment thereof, is pembrolizumab.

45. The method of claim 1, wherein the anti-PD-1 antibody, or antigen binding fragment thereof, is a pembrolizumab variant.

46. The method of claim 1, wherein the patient is administered from 320 to 420 mg of the anti-PD-1 antibody or antigen binding fragment thereof.

47-77. (canceled)

78. The method of claim 1, wherein the patient is administered 360 mg of the anti-PD-1 antibody or antigen binding fragment thereof.

79. The method of claim 1, wherein the patient is administered 370 mg of the anti-PD-1 antibody or antigen binding fragment thereof.

80. The method of claim 1, wherein the patient is administered 375 mg of the anti-PD-1 antibody or antigen binding fragment thereof.

81. The method of claim 1, wherein the patient is administered 380 mg of the anti-PD-1 antibody or antigen binding fragment thereof.

82. The method of claim 1, wherein the patient is administered 385 mg of the anti-PD-1 antibody or antigen binding fragment thereof.

83. The method of claim 1, wherein the patient is administered 390 mg of the anti-PD-1 antibody or antigen binding fragment thereof.

84. The method of claim 1, wherein the patient is administered 395 mg of the anti-PD-1 antibody or antigen binding fragment thereof.

85. The method of claim 1, wherein the patient is administered 400 mg of the anti-PD-1 antibody or antigen binding fragment thereof.

86. The method of claim 1, wherein the patient is administered 420 mg of the anti-PD-1 antibody or antigen binding fragment thereof.

87. The method of claim 1, wherein the anti-PD-1 antibody or antigen binding fragment thereof is administered as a composition comprising 130 mg/mL of the anti-PD-1 antibody or antigen binding fragment thereof.

88. The method of claim 1, wherein the anti-PD-1 antibody or antigen binding fragment thereof is administered as a composition comprising 165 mg/mL of the anti-PD-1 antibody or antigen binding fragment thereof.

89. The method of claim 87, wherein the composition further comprises 10 mM L-methionine, 10 mM histidine, pH 5.5, 7% sucrose, and 0.02% polysorbate 80.

90. The method of claim 88, wherein the composition further comprises 10 mM L-methionine, 10 mM histidine, pH 5.5, 7% sucrose, and 0.02% polysorbate 80.

91. The method of claim 1, wherein the anti-PD-1 antibody, or antigen binding fragment thereof, is administered in one or more injections.

92. The method of claim 1, wherein the anti-PD-1 antibody, or antigen binding fragment thereof, is administered in two injections.

93. A method of treating cancer in a human patient comprising subcutaneously administering to the patient about 280 mg to about 450 mg of pembrolizumab, every approximately three weeks.

94. The method of claim 93, wherein the dose is at least 1.6 times higher than a 200 mg or a 2 mg/kg dose of pembrolizumab.

95. The method of claim 94, wherein the dose is 1.9 times higher than a 200 mg or a 2 mg/kg dose of pembrolizumab.

96. The method of claim 93, wherein the bioavailability of pembrolizumab is at least 64%.

97. The method of claim 93, wherein the bioavailability of pembrolizumab is at least 66%.

98. The method of claim 93, wherein the subcutaneous administration of pembrolizumab results in a C₀ that is the same as, or greater than, the Co of a 200 mg dose or a 2 mg/kg dose of pembrolizumab administered by an intravenous (IV) route of administration.

99. The method of claim 98, wherein the subcutaneous administration of pembrolizumab results in a ratio of subcutaneous C_{trough} to IV C_{trough} of at least 1.

100. The method of claim 99, wherein the subcutaneous administration of pembrolizumab results in a ratio of subcutaneous C_{trough} to IV C_{trough} from 1.0 to 1.6.

101. The method of claim 100, wherein the dose administered by an IV route of administration is 200 mg.

102. The method of claim 93, wherein the cancer is selected from the group consisting of: melanoma, non-small cell lung cancer, small cell lung cancer, head and neck cancer, urothelial cancer, breast cancer, gastric cancer, gastroesophageal junction adenocarcinoma, multiple myeloma, hepatocellular cancer, non-Hodgkin lymphoma, primary mediastinal large B-cell lymphoma (PMBCL), renal cancer, classical Hodgkin lymphoma, mesothelioma, ovarian cancer, esophageal cancer, anal cancer, biliary tract cancer, colorectal cancer, cervical cancer, endometrial cancer, cutaneous squamous cell cancer, thyroid cancer, prostate cancer, glioblastoma, Merkel cell carcinoma, and salivary cancer.

103. The method of claim 93, wherein the patient has a tumor with a high mutational burden.

104. The method of claim 93, wherein the patient has a microsatellite instability-high (MSI-H) or mismatch repair deficient solid tumor.

105. The method of claim 102, wherein the cancer is unresectable or metastatic melanoma.

106. The method of claim 102, wherein the cancer is metastatic non-small cell lung cancer (NSCLC).

107. The method of claim 102, wherein the cancer is recurrent or metastatic head and neck squamous cell cancer (HNSCC).

108. The method of claim 102, wherein: (1) the patient is an adult and the cancer is relapsed or refractory classical Hodgkin lymphoma (cHL), or (2) the patient is a pediatric patient and cancer is refractory cHL, or cHL that has relapsed after 2 or more lines of therapy for cHL.

109. The method of claim 102, wherein the cancer is locally advanced or metastatic urothelial carcinoma.

110. The method of claim 109, wherein the patient's tumor expresses PD-L1 as measured by having a Combined Positive Score (CPS)>10.

111. The method of claim 102, wherein the patient is not eligible for platinum-containing chemotherapy or has disease progression during or following platinum-containing chemotherapy or within 12 months of neoadjuvant or adjuvant treatment with platinum-containing chemotherapy.

112. The method of claim 102, wherein the cancer is locally advanced or metastatic gastric cancer or gastroesophageal junction adenocarcinoma.

113. The method of claim 102, wherein the cancer is cervical cancer.

114. The method of claim 113, wherein the cervical cancer is recurrent or metastatic cervical cancer and the patient had disease progression on or after chemotherapy.

115. The method of claim 113, wherein the patient's tumor expresses PD-L1 as measured by a Combined Positive Score (CPS)>1.

116. The method of claim 102, wherein the cancer is primary mediastinal large B-cell lymphoma (PMBCL).

117. The method of claim 102, wherein the cancer is resected stage JIB, IIC, or III melanoma.

118. The method of claim 102, wherein the cancer is hepatocellular carcinoma.

119. The method of claim 102, wherein the cancer is renal cell carcinoma (RCC).

120. The method of claim 102, wherein the cancer is recurrent, locally advanced or metastatic Merkel cell carcinoma (MCC).

121. The method of claim 93, wherein the patient is administered from 320 to 420 mg of pembrolizumab.

122. The method of claim 93, wherein the patient is administered 360 mg of pembrolizumab.

123. The method of claim 93, wherein the patient is administered 370 mg of pembrolizumab.

124. The method of claim 93, wherein the patient is administered 375 mg of pembrolizumab.

125. The method of claim 93, wherein the patient is administered 380 mg of pembrolizumab.

126. The method of claim 93, wherein the patient is administered 385 mg of pembrolizumab.

127. The method of claim 93, wherein the patient is administered 390 mg of pembrolizumab.

128. The method of claim 93, wherein the patient is administered 395 mg of pembrolizumab.

129. The method of claim 93, wherein the patient is administered 400 mg of pembrolizumab.

130. The method of claim 93, wherein the patient is administered 420 mg of pembrolizumab.

131. The method of claim 93, wherein pembrolizumab is administered as a composition comprising 130 mg/mL of pembrolizumab.

132. The method of claim 93, wherein pembrolizumab is administered as a composition comprising 165 mg/mL of pembrolizumab.

133. The method of claim 131, wherein the composition further comprises 10 mM L-methionine, 10 mM histidine, pH 5.5, 7% sucrose, and 0.02% polysorbate 80.

134. The method of claim 132, wherein the composition further comprises 10 mM L-methionine, 10 mM histidine, pH 5.5, 7% sucrose, and 0.02% polysorbate 80.

135. The method of claim 93, wherein pembrolizumab is administered in one or more injections.

136. The method of claim 93, wherein pembrolizumab is administered in two injections.

137. The method of claim 136, wherein 1.15 mL of the composition comprising 165 mg/mL of pembrolizumab is administered in each of the two injections.

138. A kit for treating a patient with cancer, the kit comprising:
(a) a composition for subcutaneous injection comprising about 280 mg to about 450 mg of pembrolizumab, contained in one or more pre-filled syringes, and
(b) instructions for using the pembrolizumab.
